(19) United States
(12) Patent Application Publication
(10) Pub. No.: US 2024/0316210 A1
(43) Pub. Date: Sep. 26, 2024

(54) NOVEL BIOCOMPATIBLE LINKER FOR BIOORTHOGONAL APPLICATIONS

(71) Applicant: Cal Poly Corporation, San Luis Obispo, CA (US)

(72) Inventors: Holden H. Palandoken, San Luis Obispo, CA (US); Javin Oza, San Luis Obispo, CA (US); Olivia Hoffmann, San Luis Obispo, CA (US); Robert Morand Lammert, JR., San Luis Obispo, CA (US); Madeline Babros, San Luis Obispo, CA (US)

(21) Appl. No.: 18/606,989

(22) Filed: Mar. 15, 2024

Related U.S. Application Data
(60) Provisional application No. 63/490,931, filed on Mar. 17, 2023.

(51) Int. Cl. A61K 47/68

(52) U.S. Cl. CPC A61K 47/6889; A61K 47/6803

(57) ABSTRACT

The disclosure provides for methods of synthesizing a heterofunctional biolinker comprising converting a cyclohexane carboxylic acid into an carboxylic acid alkyl halide; reacting the carboxylic acid alkyl halide with a protected hydroxylamine to yield a protected alkoxyamine; converting the carboxylic acid into a reactive ester; and deprotecting the protected alkoxyamine to yield a heterofunctional biolinker capable of reacting exclusively with carbonyl groups in biomarkers and antibody drug conjugates. The biolinkers beneficially accommodate a wide variety of labels. Also disclosed are kits, compositions produced by the methods, and methods of using the same to detect a target.

### CROSS-REFERENCE TO RELATED APPLICATION

This application claims priority under 35 U.S.C. § 119 to Provisional Application U.S. Ser. No. 63/490,931, filed on Mar. 17, 2023, which is herein incorporated by reference in its entirety including without limitation, the specification, claims, and abstract, as well as any figures, tables, or examples thereof.

### TECHNICAL FIELD

The present disclosure generally relates to heterofunctional biolinkers capable of reacting exclusively with carbonyl group-containing payloads such as biomarkers, therapeutic drugs, proteins, etc., wherein the biolinkers form a covalent bond (i.e., oxime ether bond) with the carbonyl group-containing payloads at a first reactive site which contains an alkoxyamine (aka ‘aminooxy’) functionality (FIG. 1). The first reactive site can contain a single or multiple (i.e., in polymeric forms) alkoxyamine sites to accommodate the attachment of a single or multiple payloads. The other reactive site on the biolinker beneficially accommodates a wide variety of biorthogonal chemistries or labels, such as small molecules capable of fluorescence, electrochemical signal generation, and electrochemiluminescence signal generation; nucleic acids and nucleic acid-based labels such as aptamers; protein-based labels such as those capable of fluorescence, enzymatic reactions that generate color, and luminescence to support applications such as diagnostics. The other reactive site on the biolinker can also accommodate a wide variety of proteins such as antibodies to allow for the preparation of antibody drug-conjugates. Beneficially, the heterofunctional biolinkers can be synthesized in four steps using commercially available reagents.

### TECHNICAL BACKGROUND

Biological processes of life such as infections, diseases, and other medical conditions have molecular biomarkers comprising, for example, antigens, antibodies, glycoproteins, etc. The detection of these molecular biomarkers is crucial to identify the infection, type of disease, or other medical condition. Detection of biomarkers typically requires a biolinker that is capable of linking a reactive site on the biomarker and a label with a covalent bond. Depending on the nature of the reporter, a variety of detection methods such as UV-Vis absorbance, fluorescence, luminescence, electrochemistry, electrochemiluminescence, and others can be used to detect the biomarker and identify the associated infection, disease, or medical condition.

A specific example of existing detection systems includes the enzyme-linked immunosorbent assay (ELISA) which is commonly used in a variety of applications including HIV infection diagnoses and pregnancy tests.

Designing flexible biolinker systems is challenging. The biolinker must be designed to react exclusively with the reactive site on the target biomarker. Further, the price of existing biolinkers is very high. For example, 1 gram of Sulfo-SMCC (sulfosuccinimidyl 4-(N maleimidomethyl) cyclohexane-1-carboxylate), which is a commercially available biolinker, costs several thousand dollars.

There is therefore a need to develop biomarker testing capabilities for diagnostics and particularly to develop flexible biomarker testing systems capable of being easily modified to detect a wide variety of biomarkers.

Antibody drug conjugates (ADC) have shown great promise as biopharmaceuticals, especially in cancer treatments. Current antibody drug conjugates are capable of delivering a single therapeutic drug whilst the treatment of most diseases require a cocktail of different therapeutic drugs.

There is therefore a need to develop ADC technologies that can afford ADC capable of carrying multiple therapeutic drugs.

The heterofunctional biolinkers described herein are capable of ADC that can deliver a single or a cocktail of therapeutic drugs.

These and other objects, advantages, and features of the present disclosure will become apparent from the following specification taken in conjunction with the claims set forth herein.

### BRIEF SUMMARY

The present disclosure relates to methods of synthesizing a heterofunctional biolinker comprising:

(a) converting a cyclohexane carboxylic acid according to the structure

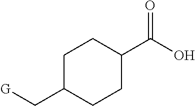

into a carboxylic acid alkyl halide, wherein G is any group that be converted into a leaving group (e.g., a halide); (b) reacting the carboxylic acid alkyl halide with a protected hydroxylamine to yield a protected alkoxyamine; (c) converting a carboxyl group on the protected alkoxyamine into a reactive ester to yield a protected alkoxyamine reactive ester; (d) deprotecting the protected alkoxyamine reactive ester; (e) optionally, adding a spacer; thereby (f) yielding a heterofunctional biolinker according to the structure:

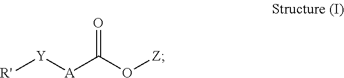

wherein R′ is —ONH₂ or absent; wherein A is a spacer; wherein Y is absent or an additional spacer; and wherein Z is a carboxylic acid activator that provides a reactive ester site.

In an embodiment, the spacer is a compound according to the formula:

G-A-Nu wherein G is a group that can be converted into an aminooxy comprising —OH, -Suc, —OPht, or an SN₂ leaving group; A is one or more monomers; and Nu is a nucleophilic group comprising —OH, —SH, or —NH₂.

In an embodiment, the spacer is a compound according to the formula:

G-A-LG wherein G is a group that can be converted into an aminooxy comprising —OH, -Suc, —OPht, or an S_{N}2 leaving group; A is one or more monomers; and LG is a leaving group comprising a tosylate, mesylate, a triflate, a triphenyl phosphate, fluoride, chloride, bromide, iodide, water, alcohol, nitrate, phosphate, thioester, amine, ammonium, carboxylate, phenoxide group, or a combination thereof.

In an embodiment, the S_{N}2 leaving group is a dinitrogen, dialkyl ether, perfluyoroalkylsulfonate, tosylate, mesylate, a triflate, a triphenyl phosphate, fluoride, chloride, bromide, iodide, water, alcohol, nitrate, phosphate, thioester, amine, ammonium, carboxylate, phenoxide, or a combination thereof.

In an embodiment, the spacer and/or the additional spacer comprises:
(i) a substituted or unsubstituted, linear or branched C₁-C₂₀ hydrocarbon;
(ii) a polyethylene glycol according to the structure

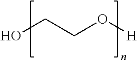

wherein n is an integer between 1 and 10,000;
(iii) an acyclic diol according to the structure

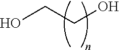

wherein n is an integer between 1 and 10,000;
(iv) a cyclic diol according to the structure

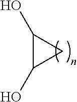

wherein n is an integer between 1 and 10,000;
(v) a poly-ONH₂ according to the structure

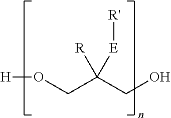

wherein n is an integer between 1 and 10,000; R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

In an embodiment, the spacer and/or the additional spacer comprises:
(i) a substituted or unsubstituted, linear or branched C₁-C₂₀ hydrocarbon;
(ii) a halogen polyethylene glycol according to the structure

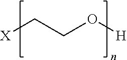

wherein n is an integer between 1 and 10,000 and X is fluoride, chloride, bromide or iodide;
(iii) a halogenated alcohol according to the structure

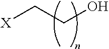

wherein n is an integer between 1 and 10,000 and X is fluoride, chloride, bromide or iodide;
(iv) a halogenated cyclic compound according to the structure

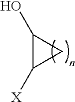

wherein n is an integer between 1 and 10,000 and X is fluoride, chloride, bromide or iodide;
(v) a poly-ONH2 according to the structure

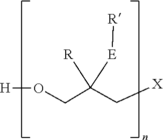

wherein n is an integer between 1 and 10,000; X is fluoride, chloride, bromide or iodide; R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

In an embodiment, the carboxylic acid alkyl halide is a compound according to the formula:

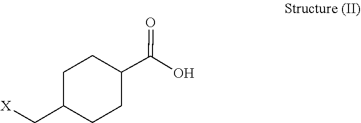

wherein X is a halide.

In an embodiment, the carboxylic acid alkyl halide comprises an alkyl fluoride, alkyl chloride, alkyl bromide, or alkyl iodide.

In an embodiment, the protected hydroxylamine is a compound according to the structure:

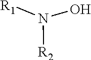

wherein R₁ is H and wherein R₂ is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

In an embodiment, the protected hydroxylamine is phenyl hydroxylamine, tolyl hydroxylamine, 2-iodophenyl hydroxylamine, N-benzyl hydroxylamine, N-methoxycarbonyl hydroxylamine, N-ethoxycarbonyl hydroxylamine, N-Boc hydroxylamine, N-CBz hydroxylamine, N-FMoc hydroxylamine, N-hydroxysuccinimide, N-hydroxyphthalimide or a combination thereof.

In an embodiment, the protected alkoxyamine is a compound according to the structure:

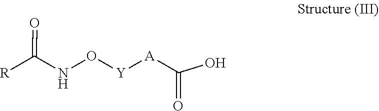

wherein R is independently selected from H, —OC(CH₃)₃, —OCH₂R where R is H, methyl, phenyl or fluorenyl, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof, the spacer A is independently selected from substituted or unsubstituted, linear or branched C₁-C₂₀ alkyl, cycloalkyl, aryl, cyclohexyl, or phenyl group or a combination thereof; and the additional spacer Y is independently selected from a substituted or unsubstituted, linear or branched C₁-C₂₀ hydrocarbon, polyethylene glycol (PEG), or a combination thereof.

In an embodiment, the protected alkoxyamine reactive ester is a compound according to the structure:

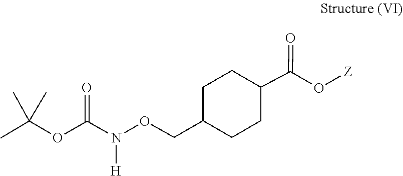

wherein Z is a carboxylic acid activator that provides a reactive ester site.

In an embodiment, the heterofunctional biolinker comprises a label L according to the structures:

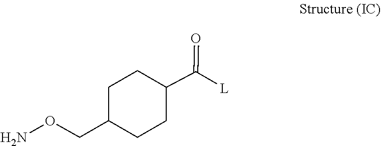

wherein L is the label; or

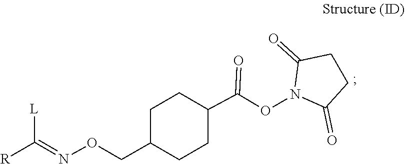

wherein L is the label.

In an embodiment, the label comprises horseradish peroxidase, a fluorescent dye, a metal, an epitope tag, a protein, an antibody, or a combination thereof.

In an embodiment, the heterofunctional biolinker is a compound according to the structure:

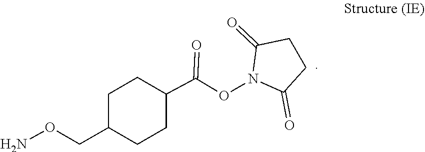 [SMILES: NOCC1CCC(C(=O)ON2C(=O)CCC2=O)CC1]

The disclosure further provides for methods of making an antibody drug conjugate compound comprising an antibody covalently attached by a heterofunctional biolinker to a drug moiety, the compound having the formula:

Ab-(H-(D)ₓ)_{y}

or a pharmaceutically acceptable salt or solvate thereof; wherein Ab is an antibody or an antigen binding fragment thereof; wherein D is the drug moiety, wherein x, which represents the one or more drugs is an integer from 1 to 20, y, which represents the number of repeating units is an integer from 1 to 10,000, and wherein H is a heterofunctional biolinker according to the structure:

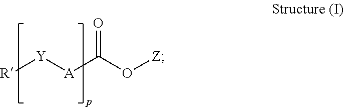

wherein R′ is —ONH₂ or absent; wherein A is a spacer; wherein Y is absent or an additional spacer; wherein Z is a carboxylic acid activator that provides a reactive ester site; and wherein p is an integer from 1 to 10,000;
wherein the method comprises:
reacting Ab with the heterofunctional biolinker to form an antibody-biolinker intermediate and reacting the antibody-biolinker intermediate with the drug moiety D to form the antibody drug conjugate; or
reacting the drug moiety D with the heterofunctional biolinker to form a drug-biolinker intermediate, and then reacting the drug-biolinker intermediate with the Ab to form the antibody drug conjugate.

In an embodiment, the antibody drug conjugate is a compound according to the structure:

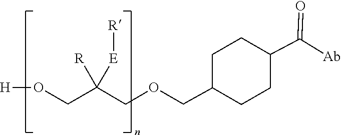

wherein R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; n is an integer between 1 and 10,000; and Ab is an antibody or an antigen binding fragment thereof.

In an embodiment, the antibody drug conjugate is a compound according to the formula:

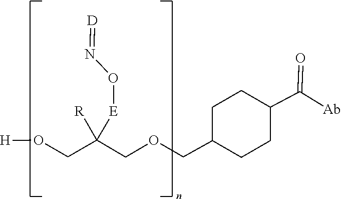

wherein Ab is an antibody or an antigen binding fragment thereof; n is an integer between 1 and 1,000; D is the drug moiety; R is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; and E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

In an embodiment, the drug moiety comprises one drug or therapeutic agent; or wherein the drug moiety comprises two or more different drugs or therapeutic agents.

In an embodiment, the drug moiety comprises a V-ATPase inhibitor, HSP90 inhibitor, IAP inhibitor, mTor inhibitor, microtubule stabilizer, microtubule destabilizer, auristatin, dolastatin, maytansinoid, methionine aminopeptidase (MetAP), CRM1 inhibitor, DPPIV inhibitor, phosphoryl transfer reaction transfer inhibitor, protein synthesis inhibitor, kinase inhibitor, CDK2 inhibitor, CDK9 inhibitor, proteasome inhibitor, kinesin inhibitor, HDAC inhibitor, DNA damaging agent, DNA alkylating agent, DNA intercalator, DNA minor groove binder, checkpoint inhibitor, immune checkpoint inhibitor, cancer growth blocker, angiogenesis inhibitor, DHFR inhibitor, or a combination thereof.

In an embodiment, the drug moiety comprises a biophysical probe, a fluorophore, a spin label, an infrared probe, an affinity probe, a chelator, a spectroscopic probe, a radioactive probe, a lipid molecule, a polyethylene glycol, a polymer, a spin label, DNA, RNA, a protein, a peptide, a surface, an antibody, an antibody fragment, a nanoparticle, a quantum dot, a liposome, a PLGA particle, a saccharide or a polysaccharide.

The disclosure also relates to heterofunctional biolinker made by the processes described herein.

Further disclosed are antibody drug conjugates made by the process of any of the processes described herein.

The disclosure also provides for heterofunctional biolinker compositions comprising: a heterofunctional biolinker according to the structure:

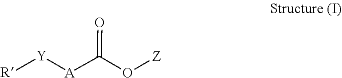

wherein R′ is —ONH₂; wherein A is a spacer; wherein Y is absent or an additional spacer; and wherein Z is a carboxylic acid activator that provides a reactive ester site.

In an embodiment, the spacer and/or the additional spacer is a compound according to the formula:

G-A-Nu

wherein G is a group that can be converted into an aminooxy (—ONH₂) comprising —OH, -Suc, —OPht, or an S_{N}2 leaving group; A is one or more monomers; and Nu is a nucleophilic group comprising —OH, —SH, or —NH₂.

In an embodiment, the spacer and/or the additional spacer is a compound according to the formula:

G-A-LG

wherein G is a group that can be converted into an aminooxy (—ONH₂) comprising —OH, -Suc, —OPht, or a combination thereof; A is one or more monomers; and LG is a leaving group.

In an embodiment, the S_{N}2 leaving group is a dinitrogen, dialkyl ether, perfluyoroalkylsulfonate, tosylate, mesylate, a triflate, a triphenyl phosphate, fluoride, chloride, bromide, iodide, water, alcohol, nitrate, phosphate, thioester, amine, ammonium, carboxylate, phenoxide, or a combination thereof.

In an embodiment, the spacer and/or the additional spacer comprises:
(i) a substituted or unsubstituted, linear or branched C₁-C₂₀ hydrocarbon;
(ii) a polyethylene glycol according to the structure

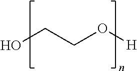

wherein n is an integer between 1 and 10,000;
(iii) an acyclic diol according to the structure

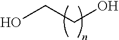

wherein n is an integer between 1 and 10,000;
(iv) a cyclic diol according to the structure

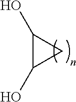

wherein n is an integer between 1 and 10,000;
(v) a poly-ONH₂ according to the structure

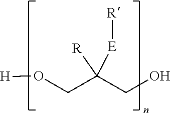

wherein n is an integer between 1 and 10,000; R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

In an embodiment, the spacer and/or the additional spacer comprises:
(i) a substituted or unsubstituted, linear or branched C₁-C₂₀ hydrocarbon;
(ii) a halogen polyethylene glycol according to the structure

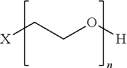

wherein n is an integer between 1 and 10,000 and X is a fluoride, chloride, bromide or iodide;
(iii) a halogenated alcohol according to the structure

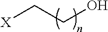

wherein n is an integer between 1 and 10,000 and X is fluoride, chloride, bromide or iodide;
(iv) a halogenated cyclic compound according to the structure

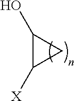

wherein n is an integer between 1 and 10,000 and X is fluoride, chloride, bromide or iodide;
(v) a poly-ONH₂ according to the structure

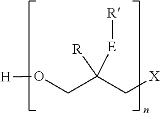

wherein n is an integer between 1 and 10,000; R′ is —ONH₂; X is fluoride, chloride, bromide or iodide; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

In an embodiment, the heterofunctional biolinker further comprises an antibody or antigen binding fragment thereof Ab and is a compound according to the structure:

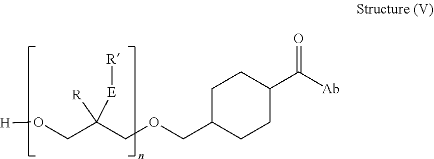

wherein R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; n is an integer between 1 and 10,000; and Ab is the antibody or an antigen binding fragment thereof.

In an embodiment, the heterofunctional biolinker further comprises an antibody or an antigen binding fragment Ab and a drug moiety D, and is a compound according to the structure:

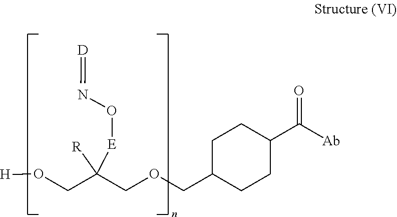

wherein Ab is the antibody or an antigen binding fragment thereof; n is an integer between 1 and 1,000; D is the drug moiety; R is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; and E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

In an embodiment, the drug moiety comprises one drug or therapeutic agent; or wherein the drug moiety comprises two or more different drugs or therapeutic agents.

In an embodiment, the composition comprises a mixture of heterofunctional biolinkers.

In an embodiment, the compositions further comprise a pharmaceutically acceptable diluent, carrier, excipient, or a combination thereof.

The disclosure further provides for methods of treating a disease in a patient in need thereof comprising: administering to the patient a therapeutically effective amount of the compositions disclosed herein.

In an embodiment, the composition is administered in a concentration of about 0.1 to about 100 mg/kg of the patient weight per dose.

In an embodiment, the composition is administered at intervals of between one week to four weeks.

In an embodiment, the disease is cancer, HIV, an autoimmune disease, a

cardiovascular disease, or a combination thereof. In an embodiment, the cancer is leukemia, lymphoma, breast cancer, cervical cancer, bladder cancer, or multiple myeloma.

The disclosure also encompasses the use of the compositions described herein in the manufacture of a therapeutic composition.

While multiple embodiments are disclosed, still other embodiments of the present disclosure will become apparent based on the detailed description, which shows and describes illustrative embodiments of the disclosure. The foregoing summary is illustrative only and is not intended to be in any way limiting. In addition to the illustrative aspects, embodiments, and features described above, further aspects, embodiments, and features of the present technology are apparent from the following drawings and the detailed description, which shows and describes illustrative embodiments of the present technology. Each feature of the technology described herein may be combined with any one or more other features of the disclosure, e.g., the methods may be used with any composition described herein. Accordingly, the drawings and detailed description are to be regarded as illustrative and not restrictive.

### BRIEF DESCRIPTION OF THE DRAWINGS

FIG. 1 shows an overview of diagnostic methods used to identify biological processes and/or medical conditions using the heterofunctional biolinkers described herein.

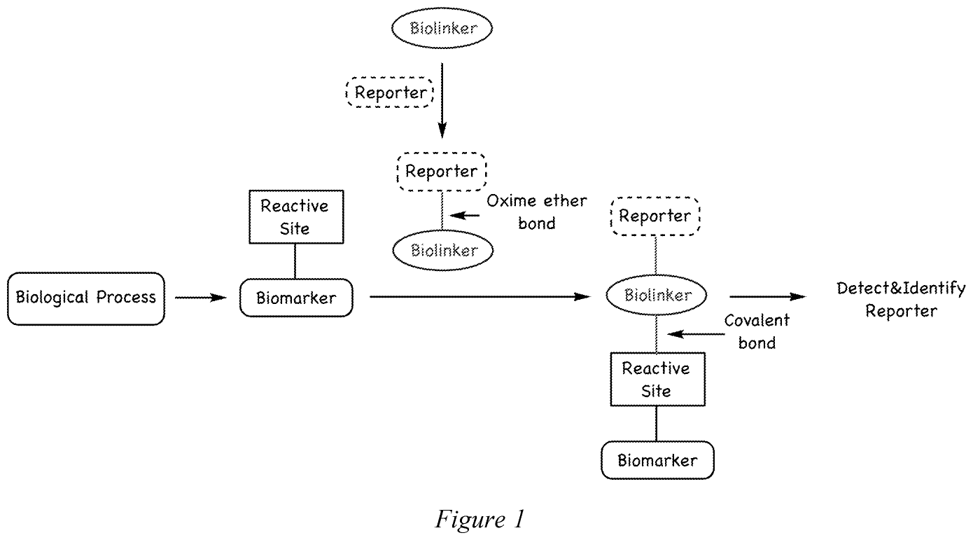

FIG. 2 shows an overview of methods for generating and using antibody drug conjugates according to the present disclosure.

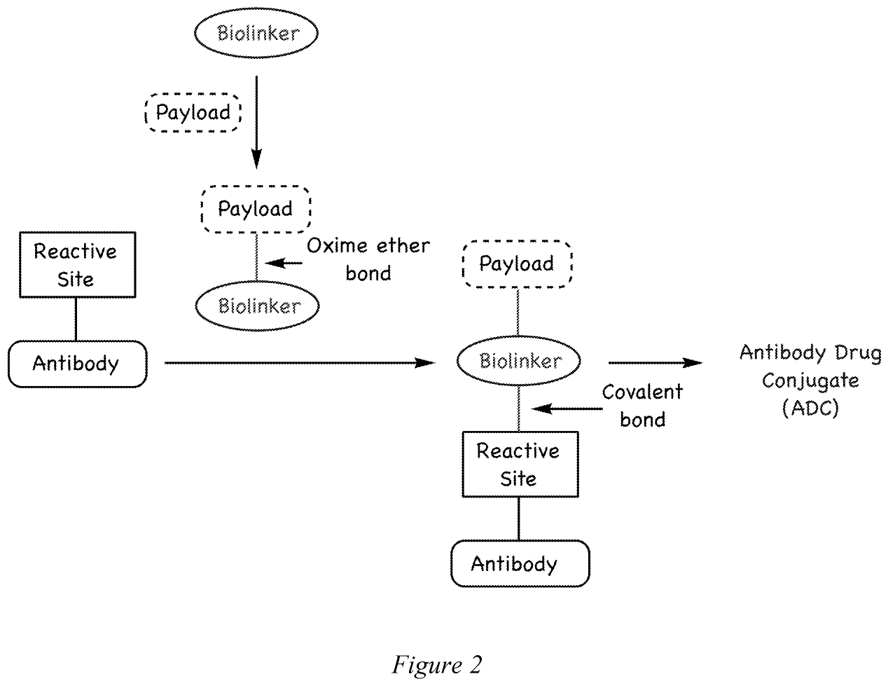

Various embodiments of the present disclosure will be described in detail regarding the drawings. Reference to various embodiments does not limit the scope of the disclosure. Figures represented herein are not limitations on the various embodiments according to the disclosure and are presented as an example illustration of the disclosure.

### DETAILED DESCRIPTION OF THE PREFERRED EMBODIMENTS

The present disclosure relates to heterofunctional biolinkers designed to react exclusively with the carbonyl reactive site on target payload such as biomarkers, therapeutic drugs, antibodies, etc.

The embodiments of this disclosure are not limited to particular types of compositions or methods, which can vary. It is further to be understood that all terminology used herein is to describe particular embodiments only and is not intended to be limiting in any manner or scope. For example, as used in this specification and the appended claims, the singular forms “a,” “an” and “the” can include plural referents unless the context indicates otherwise. Unless indicated otherwise, “or” can mean any one alone or any combination thereof, e.g., “A, B, or C” means the same as any of A alone, B alone, C alone, “A and B,” “A and C,” “B and C” or “A, B, and C” Further, all units, prefixes, and symbols may be denoted in its SI accepted form.

As used herein, the terms “comprise,” comprises,” comprising,” “include,” “includes,” and “including” can be interchanged and are to be construed as at least having the features to which they refer while not excluding any additional unspecified features.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The terms “a,” “an,” and “the” include both singular and plural referents.

The term “or” is synonymous with “and/or” and means any one member or combination of members of a particular list.

The term “about,” as used herein, refers to variation in the numerical quantity that can occur, for example, through typical measuring techniques and equipment, with respect to any quantifiable variable, including, but not limited to, mass, volume, time, temperature, pH, reflectance, whiteness, etc. Further, in practical handling procedures, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

As used herein, “substituted” refers to a group or compound having one or more hydrogen atoms replaced with one or more substituents that may be the same or different. For example, a group or compound may be substituted with a functional/organic group as described herein, (e.g., an alkyl group) in which one or more bonds to a hydrogen atom contained therein are replaced by a bond to non-hydrogen or non-carbon atoms. Substituted groups also include groups in which one or more bonds to carbon(s) or hydrogen(s) atoms replaced by one or more bonds, including double or triple bonds, to a heteroatom. Thus, a substituted group is substituted with one or more substituents, unless otherwise specified. A substituted group can be substituted with 1, 2, 3, 4, 5, or 6 substituents. Substituted ring groups include rings and ring systems in which a bond to a hydrogen atom is replaced with a bond to a carbon atom. Therefore, substituted cycloalkyl, aryl, heterocyclyl, and heteroaryl groups may also be substituted with substituted or unsubstituted alkyl, alkenyl, and alkynyl groups defined herein.

As used herein, the terms “alkyl” or “alkyl group” refers to saturated hydrocarbons having one or more carbon atoms, including straight-chain alkyl groups such as methyl, ethyl, propyl, butyl, pentyl, hexyl, heptyl, octyl, nonyl, decyl, etc. groups; cyclic alkyl groups (or “cycloalkyl” or “alicyclic” or “carbocyclic” groups) such as cyclopropyl, cyclopentyl, cyclohexyl, cycloheptyl, cyclooctyl, etc. groups; branched-chain alkyl groups such as isopropyl, tert-butyl, sec-butyl, isobutyl, etc. groups; and alkyl-substituted alkyl groups such as alkyl-substituted cycloalkyl groups and cycloalkyl-substituted alkyl groups. Unless otherwise specified, the term “alkyl” is intended to encompass both unsubstituted and substituted alkyl groups. As used herein, the term “substituted alkyl” refers to an alkyl group having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and urcido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups. In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithictane, dithiete, azolidine, pyrrolidine, pyrroline, oxolane, dihydrofuran, and furan.

The term “hydrocarbon” refers to a chemical compound containing only carbon and hydrogen. The term encompasses a wide variety of hydrocarbon compounds, the most common of which are alkanes, alkenes, alkynes, and arenes.

The term “alkane” refers to a saturated hydrocarbon according to the general formula CₙH₂ₙ₊₂. Some examples of alkanes include methane, ethane, propane, butane, and pentane.

As used herein, the terms “alkene” and “olefin” refer to unsaturated hydrocarbons that contain one or more double carbon-carbon bonds in their molecules and have the general formula CₙH₂ₙ. Examples of alkenes include ethylene, propene, and butene.

The term “alkyne” refers to unsaturated hydrocarbons that contain one or more triple carbon-carbon bonds in their molecules and have the general formula CₙH₂ₙ₋₂. Examples of alkynes include ethyne and propyne.

The term “arene” refers to a complex in which an aromatic ring is bound to a metal atom by its pi-electrons. Relatedly, an aromatic compound is one that contains a benzene ring in its molecules or has chemical properties similar to benzene.

An “alkyl halide,” also called a “haloalkane” comprises a carbon of an alkane that is bonded to one or more halogens, such as fluorine, chlorine, bromine, or iodine The presence of a halogen atom may be represented by “X” or “X-.”

As used herein, the term “alcohol” refers to an organic compound containing an —OH group and having the general formula —ROH, wherein R is absent, or represents any group or molecule attached to the —OH, i.e., the remainder of the molecule.

The term “thiol” refers to an organic compound that contains the group —SH.

The term “sulfide,” also spelled “sulphide” refers to inorganic compounds of sulfur with more electropositive elements. In particular, compounds of sulfur with nonmetals are covalent compounds, whereas metals form ionic sulfides with sulfur.

As used herein, the term “ether” refers to organic compounds containing the group —O— in their molecules, and thus having the general formula —ROR′, wherein R and R′ represent any group or molecule attached to the —O—.

The term “amine” or “amino” as used herein refers to a compound characterized by nitrogen atoms with single bonds to hydrogen and carbon and having the formula R₃₋ₓNHₓ, wherein R is a hydrocarbon or other molecule and x is an integer between 1-2. The term “amine” as used herein also refers to an independent compound and/or amine salts, wherein the hydrogen atoms attached to the nitrogen are replaced by one or more organic groups.

A “carbonyl” compound is a compound containing the carbonyl group>C═O. Aldehydes, ketones, and carboxylic acids are examples of organic carbonyl compounds. Inorganic carbonyls are complexes wherein carbon monoxide has coordinated to a metal atom or ion.

The term “aldehyde” refers to an organic compound containing the carbonyl group C═O, wherein the carbonyl group is attached to a carbon atom at the end of a carbon chain according to the formula —RCHO.

The term “ketone” refers to an organic compound containing the carbonyl group C═O, wherein the carbonyl group is attached to a carbon atom within the carbon chain, according to the formula —RCOR′.

The term “alkoxyamine” or “aminoxy” refers to an organic compound containing the group —ONH₂ and thus having a general formula of —RONH₂, wherein R represents the remainder of the molecule. The term ‘alkoxyamine’ also encompasses the protonated form of alkoxyamines, i.e., acid salts of alkoxyamines.

As used herein, the term “carboxylic acid” refers to organic compounds containing the group —COOH and thus having a general formula of —RCOOH, wherein R represents the remainder of the molecule. The term “carboxylic acid” also encompasses the deprotonated form of carboxylic acids, i.e., carboxylates and carboxylate salts.

As used herein, the term “amide” refers to organic compounds containing the group —CONH₂ or CONHR or CONR₂.

The terms “imide” and “imido group” both refer to compounds containing the group —CONHCO—.

The term “ester” refers to an organic compound formed by the reaction between alcohols and acids. Esters formed from carboxylic acids have the general formula —RCOOR′, wherein R and R′ represent the remainder of the molecule, for example, a hydrocarbon.

As used herein, the term “nitrile” refers to compounds containing the group —CN bound to another group, typically an organic group. Nitriles may be hydrolyzed to amides and carboxylic acids and can be reduced to amines

As used herein, the terms “imine” and “imino group” refer to a compound containing the group —NH— in which the nitrogen atom is part of a ring structure, or the group=NH, in which the nitrogen atom is linked to a carbon atom y a double bond.

The term “phosphate” refers to groups having phosphorous as the central atom, and typically featuring one or more P—O or P═O bonds, e.g., phosphates, diphosphates, and triphosphates.

As used herein, the term “ligand” refers generally to any molecule that binds to another molecule. In some cases, a ligand is a type of target molecule or molecule of interest.

The term “leaving group” as used herein refers to an atom or group of atoms that breaks away from the rest of the molecule, taking with it the electron pair that used to comprise the bond between the leaving group and the rest of the molecule. Nonlimiting examples of leaving groups include R—F, R—Br, R—Cl, R—I, R—(NR₃)⁺, R—(OH₂)⁺, R—OTs, R—OMs, R—OTf, R—(OPPh₃)⁺ and R—OAr. An S_{N}2 leaving group is a type of leaving group is a group that participates in a nucleophilic substitution reaction.

The terms “nucleophile” and “nucleophilic group” refer to a functional group having electron-rich atoms able to donate a pair of electrons to form a new covalent bond.

As used herein, the terms “target” and “target molecule” broadly refer to a specific molecular target that is of interest. In some cases, a target molecule is a molecule or biological material of interest. The target can be directly or indirectly representative of a biological state. It should also be understood that the target can be a fraction of a sample, such as buffy coat, a cell, such as ova, fetal material (such as trophoblasts, nucleated red blood cells, fetal red blood cells, fetal white blood cells, fetal DNA, fetal RNA, or the like), a circulating tumor cell (“CTC”), a circulating endothelial cell, an immune cell (e.g., memory T cells), a mesenchymal cell, a stem cell, a vesicle, such as an exosome, a liposome, a protein, a nucleic acid, a biological molecule, a naturally occurring or artificially prepared microscopic unit having an enclosed membrane, parasites (e.g., spirochetes, such as Borrelia burgdorferi which cause Lyme disease; malaria-inducing agents), microorganisms, viruses, inflammatory cells, or a portion of a cell culture. For example, the target can include a tumor cell from adipose tissue, an adrenal gland, bone marrow, a breast, a caudate, a cerebellum, a cerebral cortex, a cervix, a uterus, a colon, an endometrium, an esophagus, a fallopian tube, a heart muscle, a hippocampus, a hypothalamus, a kidney, a liver, a lung, a lymph node, an ovary, a pancreas, a pituitary gland, a prostate, a salivary gland, a skeletal muscle, skin, a small intestine, a large intestine, a spleen, a stomach, a testicle, a thyroid gland, or a bladder. In some cases, the biomarker is on a part of, or directly attached to the target. In other cases, the biomarker is indicative or suggestive of the biological status of the target.

As used herein, the term “label” refers generally to any molecule that is readily detectable and which makes possible makes possible the identification of another molecule, moiety, or atom (e.g., a biomarker). A label comprises at least one reactive group that forms a covalent bond with the biolinkers described herein. For example, many dyes comprise NHS as a reactive group, while other cell particles and proteins have COOH and maleimide as reactive groups, respectively. The term “label” is used interchangeably with and is intended to encompass molecules referred to as “reporters.”

As used herein, the term “antibody” refers to an immunoglobulin that specifically binds to and is thereby defined as complementary with a particular spatial and polar organization of another molecule. The antibody may be monoclonal or polyclonal and may be prepared by techniques that are well known in the art such as immunization of a host and collection of sera (polyclonal), or by preparing continuous hybrid cell lines and collecting the secreted protein (monoclonal), or by cloning and expressing nucleotide sequences or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM. Functional antibody fragments may include portions of an antibody capable of retaining binding at similar affinity to full-length antibody (for example, Fab, Fv, and F(ab′)₂, or Fab′). In addition, aggregates, polymers, and conjugates of immunoglobulins or their fragments may be used where appropriate so long as binding affinity for a particular molecule is substantially maintained.

“Complementarity-determining domains” or “complementary-determining regions (“CDRs”) interchangeably refer to the hypervariable regions of light chain variable region (VL) and heavy chain variable region (VH). The CDRs are the target protein-binding site of the antibody chains that harbors specificity for such target protein. There are three CDRs in each human VL or VH, constituting about 15-20% of the variable domains. The CDRs are structurally complementary to the epitope of the target protein and are thus directly responsible for the binding specificity. The remaining stretches of the VL or VH, the so-called framework regions, exhibit less variation in amino acid sequence. The positions of the CDRs and framework regions can be determined using various well known definitions and resources available to one skilled in the art. Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

The term “antigen binding fragment” as used herein refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment, which consists of a VH domain; and an isolated complementarity determining region (CDR), or other epitope-binding fragments of an antibody. Antigen binding fragments can also be incorporated into single domain antibodies, maxibodies, minibodies, nanobodies, intrabodies, diabodies, triabodies, tetrabodies, v-NAR and bis-scFv. Antigen binding fragments can be grafted into scaffolds based on polypeptides such as fibronectin type III (Fn3) as described in U.S. Pat. No. 6,703,199, which is herein incorporated by reference in its entirety. Antigen binding fragments can be incorporated into single chain molecules comprising a pair of tandem Fv segments (VH-CH1-VH-CH1) which, together with complementary light chain polypeptides, form a pair of antigen binding regions as described, for example, in U.S. Pat. No. 5,641,870, which is herein incorporated by reference in its entirety.

The term “monoclonal antibody” or “monoclonal antibody composition” as used herein refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis. Human antibodies can include amino acid residues not encoded by human sequences (e.g., mutations introduced by random or site-specific mutagenesis in vitro or by somatic mutation in vivo, or a conservative substitution to promote stability or manufacturing.

The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

As used herein, the term “biomarker” refers generally to any biological or biochemical measure of a normal biological process, pathogenic process, or response to an exposure or intervention, including a therapeutic intervention. Types of biomarkers include those which are molecular, histologic, radiographic or encompass physiological characteristics. A description of a biomarker can include one or more of the biomarker names, the source/matrix, the measurable characteristic(s), and the analytic method used to measure the biomarker. The biomarker may be a single characteristic or a panel of multiple characteristics. Categories of biomarkers include susceptibility/risk biomarkers, diagnostic biomarkers, monitoring biomarkers, response biomarkers, predictive biomarkers, prognostic biomarkers, and safety biomarkers. Further discussion of the categories and examples of biomarkers is found in the BEST (Biomarkers, EndpointS, and other Tools) Resource published by the FDA-NIH Biomarker Working Group (last updated Nov. 29, 2021) and is herein incorporated by reference in its entirety.

A “diagnostic biomarker” is a biomarker used to detect or confirm the presence of a disease or condition of interest, or to identify individuals with a subtype of the disease.

A “monitoring biomarker” is a biomarker measured repeatedly for assessing the status of a disease or medical condition or for evidence of exposure to (or effect of) a medical product or an environmental agent.

A “predictive biomarker” is a biomarker used to identify individuals who are more likely than similar individuals without the biomarker to experience a favorable or unfavorable effect from exposure to a medical product or an environmental agent.

A “prognostic biomarker” is a biomarker used to identify the likelihood of a clinical event, disease recurrence, or progression in patients who have the disease or medical condition of interest.

A “response biomarker” is a biomarker used to show that a biological response, potentially beneficial or harmful, has occurred in an individual who has been exposed to a medical product or an environmental agent. Response biomarkers include, for example, pharmacodynamic biomarkers, which are response biomarkers that indicate biologic activity of a medical product or environmental agent without necessarily drawing conclusions about efficacy or disease outcome or necessarily linking this activity to an established mechanism of action. Relatedly, surrogate endpoint biomarkers are a type of response biomarker that is an endpoint used in clinical trials as a substitute for a direct measure of how a patient feels, functions, or survives. A surrogate endpoint does not measure the clinical benefit of primary interest in and of itself, but rather is expected to predict that clinical benefit or harm based on epidemiologic, therapeutic, pathophysiologic, or other scientific evidence.

A “safety biomarker” is a biomarker measured before or after exposure to a medical product or an environmental agent to indicate the likelihood, presence, or extent of toxicity as an adverse effect.

The terms “susceptibility biomarker” and “risk biomarker” refer to any biomarker that indicates the potential for developing a disease or medical condition in an individual who does not currently have clinically apparent disease or the medical condition.

As used herein, the terms “reactive target,” “reactive group,” and “active group” refer to an atom, associated group of atoms, and/or a particular site on a molecule that is intended, or may be reasonably expected, to undergo a chemical reaction and in particular exhibit a characteristic reactivity with distinctive chemical behavior. Many such reactive groups are or include functional/organic groups such as saturated or unsaturated hydrocarbons (e.g., alkanes, alkenes, alkynes, aromatics), halides, alcohols, ethers, sulfides, amines, carbonyls (e.g., aldehydes and ketones), carboxylic acids and acid derivatives (e.g., amides, esters, acid anhydrides, acyl phosphates, and acid chlorides), nitriles, imines, phosphates, thiols, and imides. A molecule may have one or more than one reactive groups.

As used herein, the terms “chemical specificity” and “specificity” generally refer to the binding ability between the reactive group(s) of the biomarker and the reactive group(s) of the biolinker, along with the strength of binding between the same. The type of specificity can be classified as non-specific or specifiC Specific binding refers to an occurrence of a ligand binding to the receptor of interest, while non-specific binding (NSB) refers to an occurrence of a ligand binding to other or unintended sites.

The methods, systems, apparatuses, and compositions disclosed herein may comprise, consist essentially of, or consist of the components and ingredients described herein as well as other ingredients not described herein. As used herein, “consisting essentially of” means that the methods, systems, apparatuses, and compositions may include additional steps, components, or ingredients, but only if the additional steps, components, or ingredients do not materially alter the basic and novel characteristics of the claimed methods, systems, apparatuses, and compositions.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

The “scope” of the present disclosure is defined by the claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

### Heterofunctional Carbonyl Biolinkers

Disclosed herein are heterofunctional carbonyl biolinkers according to the structure:

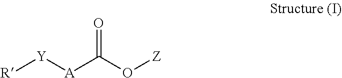

wherein R′ is —ONH₂ or absent; wherein A is a spacer; wherein Y is absent or an additional spacer; and wherein Z is a carboxylic acid activator that provides a reactive ester site.

In a preferred embodiment, the heterofunctional biolinker is a compound according the structure:

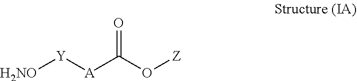

wherein A is a spacer; Y is absent or is an additional spacer and wherein the spacer and/or additional spacer comprise a substituted or unsubstituted, linear or branched C₁-C₂₀ hydrocarbon, polyethylene glycol (PEG), or a combination thereof; and wherein Z is a carboxylic acid activator comprising a diimidyl group such as succinimidyl and phthalimidyl or substituted or unsubstituted, linear or branched C₁-C₂₀ alkyl, alkenyl, alkynyl, cycloalkyl, aryl, cyclohexyl, or benzyl group or a combination thereof; and wherein Z provides a reactive carbonyl site.

The heterofunctional carbonyl biolinkers demonstrate exclusivity (i.e., exclusive binding) with carbonyl-containing payloads such as biomarkers, therapeutic drugs, antibodies, etc. Specifically with aldehyde-and ketone-containing biomarkers, detection of aldehydes and ketones can occur in a variety of raw, purified, and complex samples, including saliva, blood, urine, sweat, cell lysates, waste water, agricultural samples, chemical reactions, and others. The biolinkers disclosed herein form an oxime ether bond between the reactive site (i.e., aldehyde or ketone carbonyl site) of the payload such as a biomarker, therapeutic drug, antibody, etc. and the alkoxyamine site of the biolinker and form an amide bond between the reactive carbonyl site located on the activated carboxylic acid of the biolinker and the label or non-label in aqueous media. The ease with which alkoxyamines (RONH₂) condense with aldehydes and ketones has prompted their widespread use in labeling and targeting liposome, bacterial and mammalian cell surfaces as well as chemoselectively ligating small molecule “recognition elements” onto polyfunctional substrates. Beneficially, the heterofunctional biomarkers described herein can accommodate a wide variety of labels, such as horse radish peroxidase (HRP); small molecules capable of fluorescence, electrochemical signal generation, and electrochemiluminescence; nucleic acids and nucleic acid-based labels such as aptamers; protein-based labels such as those capable of fluorescence, enzymatic reactions that generate color, and luminescence and non-label molecules such as antibodies, coenzyme A, etc. The heterofunctional biomarkers can also beneficially distinguish between reducing and non-reducing reagents.

In an embodiment, the biolinker is a compound according to the structure:

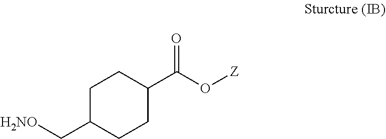

wherein Z is a carboxylic acid activator that provides a reactive ester site. In an embodiment, Z comprises a diimidyl group such as succinimidyl and phthalimidyl or substituted or unsubstituted, linear or branched C₁-C₂₀ alkyl, alkenyl, alkynyl, cycloalkyl, aryl, cyclohexyl, or benzyl group or a combination thereof.

In some embodiments, the heterofunctional biolinker comprises one or more labels as discussed herein, and which may be represented by the structures:

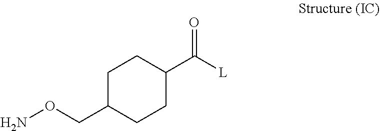

wherein L is a label; or

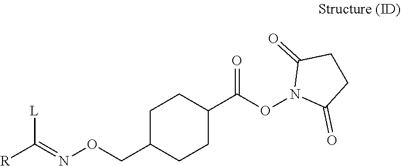

wherein L is a label.

In a still further embodiment, the biolinker is a compound according to the structure:

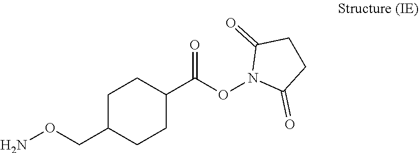 [SMILES: NOCC1CCC(C(=O)ON2C(=O)CCC2=O)CC1]

### Spacers

The biolinkers described herein optionally comprise a spacer. Functionality includes MS-cleavable groups, isotope-coding, enrichment handles, and related capture and release groups. Often different spacer arm lengths are required because steric effects dictate the distance between potential reaction sites for cross-linking. For protein-protein interaction studies, a cross-linker with a short (4-8 Å) spacer arm is preferably used first and the degree of cross-linking is determined. A cross-linker with a longer spacer arm may then be used to optimize cross-linking efficiency. Short spacer arms are often used in intramolecular cross-linking studies, and intermolecular cross-linking is favored with a cross-linker containing a long spacer arm. Alternatively, if the spacer arm is too long, intermolecular cross-linking can occur.

Spacers may have any suitable composition. Examples include, but are not limited to, hydroxylamine-cleavable spacers, acid-cleavable spacers, spacers cleaved by sodium dithionate reduction, spacers cleaved by reduction, photocleavable spacers, or a combination thereof. More particularly, suitable spacers include, but are not limited to, a C₁-C₂₀ branched or linear hydrocarbon chain, polyethylene glycol (PEG), a pinacol ester, azobenzene, disulfide, an acetal group, or a combination thereof.

Spacers may be installed on the biolinkers described herein via any suitable mechanism and at any desirable step in the process of synthesizing the biolinker. For example, one or more spacers may be installed on Structure (IIA) described herein using S_{N}2 as follows:

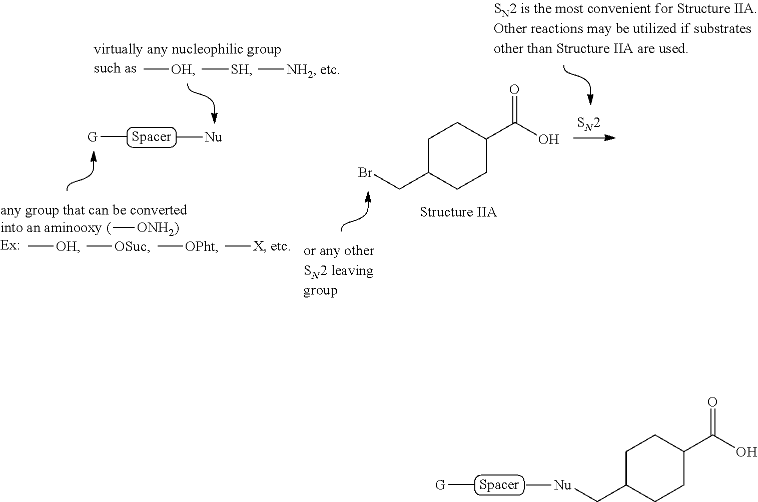

Some examples of G-Spacer-Nu include, without limitation:

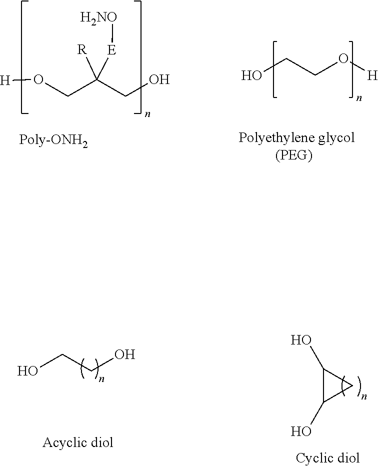

Alternatively, one or more spacers may be installed as described herein using S_{N}2 as follows:

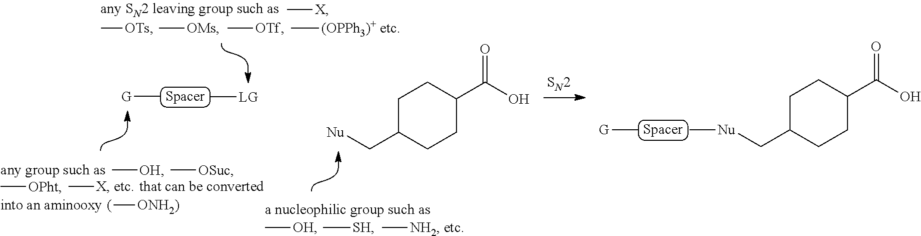

Some examples of G-Spacer-LG include, without limitation:

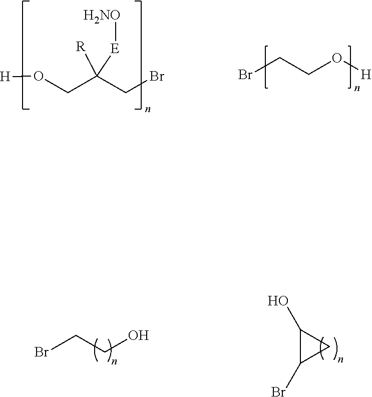

wherein R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; n is an integer between 1 and 10,000; and Ab is an antibody or an antigen binding fragment thereof.

In some embodiments, spacers such as Poly-ONH₂ provide several alkoxyamine (aka ‘aminooxy’) sites to bond with more than one type of payload such as a therapeutic compound (e.g., drug). Specifically, this leads to an antibody drug conjugate (ADC) with more than one type of therapeutic compound bound to it. An ADC with multiple alkoxyamine (aka ‘aminooxy’) sites can be synthesized according to Scheme 1:

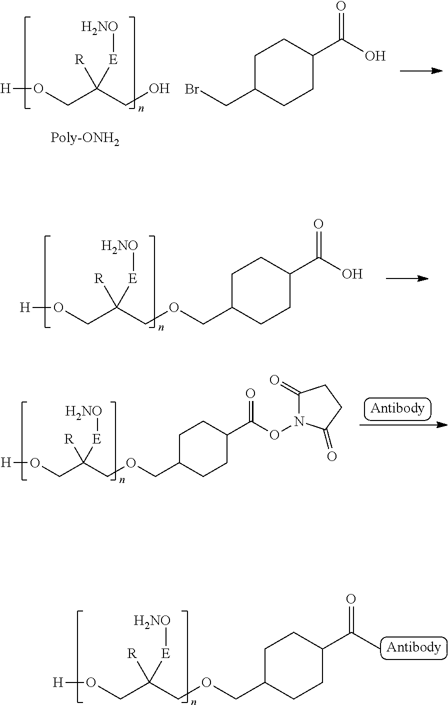

Scheme 1 illustrates the synthesis of an antibody with multiple alkoxyamine (i.e., ‘aminooxy’) sites to accommodate the attachment of more than one type of payload such as a therapeutic compound with an oxime ether bond.

An ADC bonded to more than one type of therapeutic compound can be synthesized according to Scheme 2:

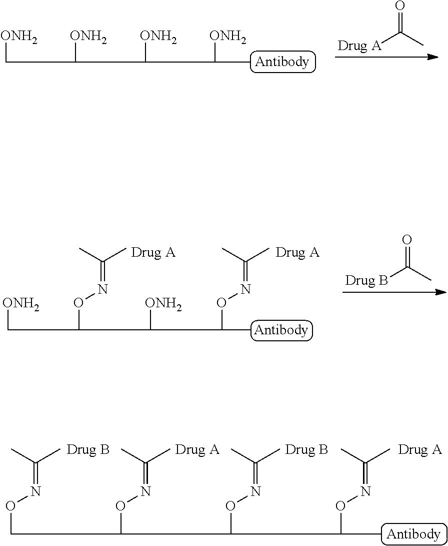

Scheme 2 illustrates the synthesis of an antibody bonded to more than one type of therapeutic compound with an oxime ether bond. Further discussion of ADCs and therapeutic compounds is provided herein.

Alternatively, Scheme 3 illustrates one example of the synthesis of an antibody modified to possess a carbonyl site (using nonstandard amino acids, or post-translational modifications) bonded to the alkoxyamine site with the oxime ether bond whilst the payload such as therapeutic compound is bonded to the reactive carboxylic acid site:

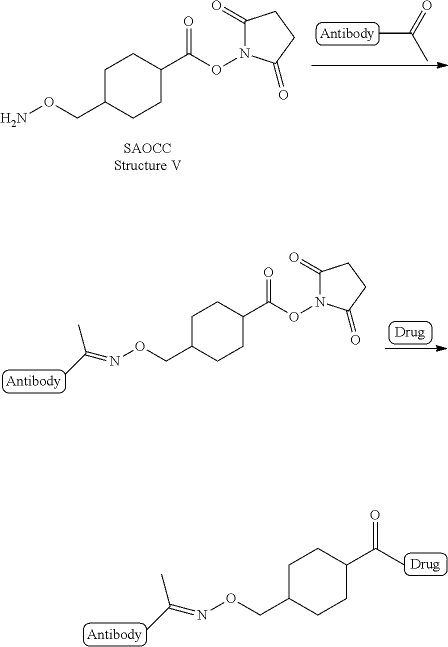

Accordingly, preferred antibody drug conjugates include, but are not limited to, those of the following structures:

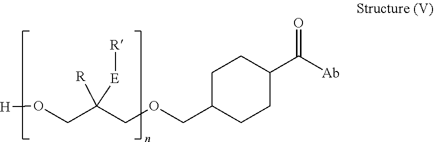

wherein R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; n is an integer between 1 and 10,000; and Ab is an antibody or an antigen binding fragment thereof and/or;

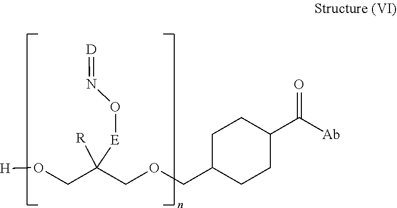

wherein Ab is an antibody or an antigen binding fragment thereof; n is an integer between 1 and 1,000; D is a drug moiety; R is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; and E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

### Labels

The biolinkers described herein preferably include at least one reporter. A label is a molecule or compound capable of detection, thereby permitting the identification, quantification, distribution mapping, or pathway mapping of another molecule, compound, or atom (e.g., a biomarker). Labels can be incorporated into, coupled to, or associated with, compounds, detection compounds, or capture compounds (such as compounds to be bound to proteins). A label can include, for example, a fluorescent dye, a member of binding pair, such as biotin/streptavidin, a metal (e.g., gold), or an epitope tag that can specifically interact with a molecule that can be detected, such as by producing a colored substrate or fluorescence. Many other types of labels and signals, and many other principals of signal detection and known and can also be used, some of which are described herein. For example, labels (and other compounds and components) can be detected using nuclear magnetic resonance, electron paramagnetic resonance, surface enhanced raman scattering, surface plasmon resonance, fluorescence, phosphorescence, chemiluminescence, resonance raman, microwave, photometry, mass spectrometry, or a combination.

Each label has at least one reactive group capable of forming a covalent bond with the biolinkers. Examples of reactive groups include, but are not limited to, a maleimide, NHS (succinimidyl), iodoacetyl, bromoacetyl, sulfonyl chloride, aldehyde, chloromethyl, alkyne, thiol, azide, amine group, or a combination thereof.

Examples of suitable types of labels include, for example, fluorescent dyes (also known herein as fluorochromes and fluorophores), chromophores, and enzymes that react with colorimetric substrates (e.g., horseradish peroxidase). Fluorophores are compounds or molecules that luminesce. Typically, fluorophores absorb electromagnetic energy at one wavelength and emit electromagnetic energy at a second wavelength.

Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactmomycm D (7-AAD); 7-Hydroxy-4-1 methylcoumariii; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Acquorin (Photoprotein); AFPs-AutoFluorescent Protein-(Quantum Biotechnologies) see sgGFP, sgBFP; Alexa Fluor 350; Alexa Fluor 430; Alexa Fluor 488; Alexa Fluor 532; Alexa Fluor 546; Alexa Fluor 568; Alexa Fluor 594; Alexa Fluor 633; Alexa Fluor 647; Alexa Fluor 660; Alexa Fluor 680; Alizarin Complexon; Alizarin Red; Allophycocyanin (APC), including APC/Cy 5.5 and APC/Cy 7; AMC, AMCA-S; Ammomethylcoumarin (AMCA); AMCA-X; Aminoactinomycin D; Ammocoumarin; Anilin Blue; Anthrocyl stearate; APTRA-BTC; APTS; Astrazon Brilliant Red 4G; Astrazon Orange R; Astrazon Red 6B; Astrazon Yellow 7 GLL; Atabrine; ATTO-TAG CBQCA; ATTO-TAG FQ; Atto 390, Atto 425, Atto 465, Atto 488, Atto 532, Atto 565, Atto 590, Atto 594, Atto 610, Atto 61, Atto 620, Atto 633, Atto 637, Atto 655, Atto 680, Atto 700, Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO-1; BOBO-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X5 SE; Bodipy TR; Bodipy TR ATP; Bodipy TR-X SE; BO-PRO-1; BO-PRO-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coclenterazine; Coclenterazine ep; Coelenterazine f; Coclenterazine fcp; Coclenterazine h; Coclenterazine hep; Coclenterazine ip; Coclenterazine n; Coclenterazine O; Coumarin Phalloidin; C-phycocyanine, Cyanine dye 3.5, Cyanine dye 5, Cyanine dye 5.5, Cyanine dye 7, fluorescein, PerCP/Cy 5.5, R/B/C-Phycocrythrin, streptavidin, Allophycocyanin (A-PC), C-Phycocyanin (C-PC), R-Phycocyanin (R-PC); CPM I Methylcoumarin; CTC; CTC Formazan; Cy2; Cy3.1 8; Cy3.5; Cy3; Cy5.1 8; Cy5.5; Cy5; Cy7, PE/Cy 5, PE/Cy 5.5, PE/Cy 7; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS; DiA (4-Di 16-ASP); Dichlorodihydrofluorescein Diacetate (DCFH); DiD-Lipophilic Tracer; DiD (DilC18(5)); DE)S; Dihydorhodamine 123 (DHR); DiI (DiICl 8(3)); I Dinitrophenol; DiO (DiOC18(3)); DiR; DiR (DilC18(7)); DM-NERF (highpH); DNP; Dopamine; DsRed; DTAF; DY-630-NHS; DY-635-NHS; EBFP; ECFP; EGFP; ELF 97; Eosin; Erytihrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43; FM 4-46; Fura Red (high pH); Fura Red/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; hidodicarbocyanine (DiD); Indotricarbocyanine (DiR); Intrawhite Cf; JC-I; JO JO-I; JO-PRO-I; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-I; LO-PRO-I; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (niBBr-GSH); Monochlorobimane; MPS (Methyl Green Pyronine Stilbene); NBD; NBD Amine; Nile Red; Nitrobenzoxedidole; Noradrenaline; Nuclear Fast Red; Nuclear Yellow; Nylosan Brilliant lavin E8G; Oregon Green; Oregon Green 488; Oregon Green 500; Oregon Green 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-I; PO-1 PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP (super glow BFP); sgGFP (super glow GFP); SITS (Primuline; Stilbene Isothiosulphonic Acid); SNAFL calcein; SNAFL-I; SNAFL-2; SNARF calcein; SNARFl; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red; Texas Red-X conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofhior White); TIER; TO-PRO-I; TO-PRO-3; TO-PRO-5; TOTO-I; TOTO-3; Tricolor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-I; YO-PRO 3; YOYO-1;YOYO-3; Sybr Green; Thiazole orange, or a combination thereof.

Other suitable types of labels include molecular barcodes, mass labels, and labels detectable by nuclear magnetic resonance, electron paramagnetic resonance, surface enhanced raman scattering, surface plasmon resonance, fluorescence, phosphorescence, chemiluminescence, resonance raman, microwave, photometry, mass spectrometry, or a combination. Mass labels are compounds or moieties that have, or which give the labeled component, a distinctive mass signature in mass spectroscopy. Mass labels are useful when mass spectroscopy is used for detection. Examples of mass labels include peptide nucleic acids and carbohydrates.

Epitopes can also be used as labels. An epitope, which is a portion of a molecule to which an antibody binds, can be composed of sugars, lipids, or amino acids. Epitope tags are useful for the labeling and detection of proteins when an antibody to the protein is not available. Epitope tags generally range from 10 to 15 amino acids long and are designed to create a molecular handle for the protein. An epitope tag can be placed anywhere within the protein. Any short stretch of amino acids known to bind an antibody could become an epitope tag. Useful epitope tags include c-myc (a 10 amino acid segment of the human protooncogene myc), hemagglutinin (HA) protein, Hisβ, Green fluorescent protein (GFP), digoxigenin (DIG), Peridinin-chlorophyll-protein complex (PerCP), and biotin. Fluorescent dyes, such as those described herein, can also be used as epitope tags.

### Carbonyl-Containing Biomarkers

As the biolinkers described herein selectively react with aldehydes and ketones, the biomarker is preferably a marker containing a ketone and/or an aldehyde.

In one embodiment, the biomarker has an aldehyde, for example, a C₂₋₂₀ aldehyde, preferably a C₂₋₁₄ aldehyde. Other specific aldehyde biomarkers include, but are not limited to, enanthaldehyde, octanal, ethylo benzene formaldehyde, 2-tolylaldehyde, 3-tolylaldehyde, 3-methylbenzaldehyde, and aldehyde dehydrogenase (ALDH). In another embodiment, the biomarker has a ketone, preferably a C₃₋₂₀ ketone, including a C₃₋₁₄ ketone, and a C₆₋₁₂ ketone. Specific examples of ketone biomarkers include, but are not limited to, acetone, 2-butanone, 2-pentanone, 2-heptanone, 4-heptanone, a methyl-hexyl ketone, a 3-octene-2-ketone, a methyl n-heptyl ketone, 2-decanones, cyclohexanone, acetophenone, and piperitone.

Carbohydrate biomarkers are an important class of aldehyde-and ketone-containing biomarkers. In particular, carbohydrate-associated antigens (CAAs) function as effective biomarkers for cancer diagnosis and therapeutic targets. Examples of CAAs include, but are not limited to, Tn and TF antigens, globo H, sialyl Lewis X (sLex), Lewis Y (Ley), and polysialic acids. For example, antigens Tn (CD175) and globo H are the most commonly found CAAs on cancer cells. Others include sTn, di-sTF, globo H, sLex, sLea, SLX, ST-439, galectin-1, galcctin-3, galectin-7, and 9, BCM, MUC1, LPA, CEA, M344, MCA, and PMA.

Carbohydrate biomarkers, specifically CAAs associated with the detection of infectious diseases include GP120/polymannose (for HIV) and LPS/endotoxins (for bacterial infections).

Other CAAs that function as biomarkers include but are not limited to Glycated Hb (GlcHb) and galectin-3 (for cardiovascular disease), Le^{x}, ABO blood group antigens, and α-Gal-α-Gal (for inflammation and immune responses), stage-specific biomarkers such as Le^{x}, SSEA-1,3 and 4, fucose and sialic acid (indicative of stem cell differentiation and embryo development); and glycosphingolipids such as gangliosides, cerebrosides, and globosides, etc.

Further discussion of suitable biomarkers is found, for example in Huddle, B.C et al., Structure-Based Optimization of a Novel Class of Aldehyde Dehydrogenase 1A (ALDH1A) Subfamily-Selective Inhibitors as Potential Adjuncts to Ovarian Cancer Chemotherapy. J MED CHEM. 2018, 61(19):8754-8773; Dai, Chaofeng et al., Carbohydrate biomarker recognition using synthetic lectin mimics, PURE APP. CHEM. 2012 84(11):2479-2498; and Cheng, Yun Feng, et al., Carbohydrate biomarkers for future disease detection and treatment, SCI. CHINA 2010 (53)1:3-20, which are herein incorporated by reference in its entirety.

Malondialdehyde and 4-hydroxy-2-nonenal have been identified as biomarkers of several diseases such as asthma, cardiovascular disease, and bronchiectasis induced by oxidative stress and inflammation. See references: Khoubnasabjafari, M. et al., Critical Review of Malondialdehyde Analysis in Biological Samples. Curr Pharm Anal 2016, 12 (1), 4-17; Antus, B. et al., Monitoring oxidative stress during chronic obstructive pulmonary disease exacerbations using malondialdehyde. Respirology 2014, 19 (1), 74-79; Casimirri, E. et al., Biomarkers of oxidative-stress and inflammation in exhaled breath condensate from hospital cleaners. Biomarkers 2016, 21 (2), 115-122.

### Antibody Drug Conjugates, Conjugates and Preparation Thereof

The biolinker described herein allows for the chemistry-selective conjugation of two or more distinct small molecules and biologics. The biorthogonal nature of the biolinker supports conjugation to an antibody and a therapeutic compound or drug. Antibody drug conjugates (“ADCs”) may be used for the local delivery of cytotoxic agents or other therapeutic compounds in the treatment of cancer or other diseases. ADCs beneficially allow the targeted delivery of a therapeutic compound/moiety where maximum efficacy may be achieved with minimal toxicity. Further discussion of methods of making and optimizing ADCs is found in Grunewald, et al. (2017) which is herein incorporated by reference in its entirety.

The antibody could be conjugated to position Z of structure (I), or via the alkoxyamine if the antibody is modified using nonstandard amino acids, or post-translational modifications. The drug can be conjugated to either position Z of structure (I), or via the alkoxyamine. The resulting entities would support therapeutic applications.

In another embodiment, the heterofunctional biolinker contains multiple alkoxyamines, supporting the conjugation of multiple drugs, for example according to the structure:

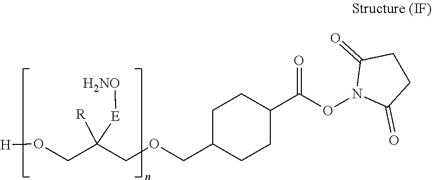

More particularly, in an embodiment where the heterofunctional biolinker contains multiple alkoxyamines, a therapeutic compound (e.g., a drug) or any payload with an aldehyde or ketone carbonyl site may be covalently attached to the heterofunctional biolinker at the site of the alkoxyamines:

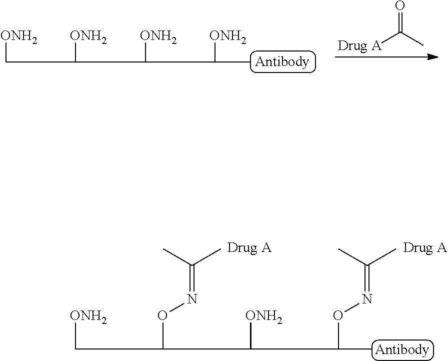

In an embodiment, a second therapeutic compound having a carbonyl group may be covalently attached to the heterofunctional biolinker at the site of the alkoxyamines:

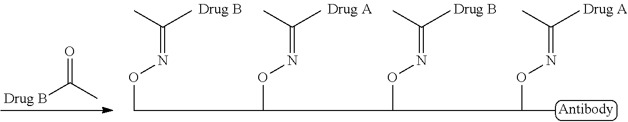

More generally, the present disclosure relates to antibody drug conjugates or a pharmaceutically acceptable salt thereof according to the formula:

Ab-(H-(D)ₓ)_{y}

wherein Ab is an antibody or antigen binding fragment thereof; H is the heterofunctional biolinker described herein, D is a drug moiety or therapeutic compound, x is an integer from 1 to 20; and y is an integer from 1 to 10,000.

The therapeutic compound or drug moiety may be any suitable moiety or compound providing a desirable therapeutic or diagnostic effect, for example, an anti-cancer, anti-inflammatory, anti-infective (e.g., anti-fungal, antibacterial, anti-parasitic, anti-viral), or an anesthetic agent. In certain aspects, a drug moiety is selected from a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder and a DHFR inhibitor. Methods for attaching each of these to a linker compatible with the antibodies and method of the present disclosure are known in the art. In addition, the therapeutic compound can be a biophysical probe, a fluorophore, a spin reporter, an infrared probe, an affinity probe, a chelator, a spectroscopic probe, a radioactive probe, a lipid molecule, a polyethylene glycol, a polymer, a spin reporter, DNA, RNA, a protein, a peptide, a surface, an antibody, an antibody fragment, a nanoparticle, a quantum dot, a liposome, a PLGA particle, a saccharide or a polysaccharide.

Further, the antibodies, antibody fragments (e.g., antigen binding fragments) or functional equivalents of the present disclosure may be conjugated to a drug moicty that modifies a given biological response. Drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin, a protein such as tumor necrosis factor, α-interferon, β-interferon, nerve growth factor, platelet derived growth factor, tissue plasminogen activator, a cytokine, an apoptotic agent, an anti-angiogenic agent, or, a biological response modifier such as, for example, a lymphokine.

In one aspect, the antibodies, antibody fragments (e.g., antigen binding fragments) or functional equivalents of the present disclosure are conjugated to a drug moiety, such as a cytotoxin, a drug (e.g., an immunosuppressant) or a radiotoxin. Examples of cytotoxin include but are not limited to, taxanes (sec, e.g., International (PCT) Patent Application Nos. WO 01/38318 and PCT/US03/02675), DNA-alkylating agents (e.g., CC-1065 analogs), anthracyclines, tubulysin analogs, duocarmycin analogs, auristatin E, auristatin F, maytansinoids, and cytotoxic agents comprising a reactive polyethylene glycol moiety (see, e.g., Sasse et al., J. Antibiot. (Tokyo), 53, 879-85 (2000), Suzawa et al., Bioorg. Med. Chem., 8, 2175-84 (2000), Ichimura et al., J. Antibiot. (Tokyo), 44, 1045-53 (1991), Francisco et al., Blood (2003) (electronic publication prior to print publication), U.S. Pat. Nos. 5,475,092, 6,340,701, 6,372,738, and 6,436,931, U.S. Patent Application Publication No. 2001/0036923 Al, Pending U.S. patents application Ser. Nos. 10/024,290 and 10/116,053, and International (PCT) Patent Application No. WO 01/49698), taxon, cytochalasin B, gramicidin D, ethidium bromide, emetine, mitomycin, etoposide, tenoposide, vincristine, vinblastine, t. colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, anti-metabolites (e.g., methotrexate, 6-mercaptopurine, 6-thioguanine, cytarabine, 5-fluorouracil decarbazine), ablating agents (e.g., mechlorethamine, thiotepa chlorambucil, meiphalan, carmustine (BSNU) and lomustine (CCNU), cyclophosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). (See e.g., Seattle Genetics U520090304721).

Other examples of cytotoxins that can be conjugated to the antibodies, antibody fragments (antigen binding fragments) or functional equivalents of the present disclosure include duocarmycins, calicheamicins, maytansines and auristatins, and derivatives thereof.

Various types of cytotoxins, linkers and methods for conjugating therapeutic agents to antibodies are known in the art, sec, e.g., Saito et al., (2003) Adv. Drug Deliv. Rev. 55:199-215; Trail et al., (2003) Cancer Immunol. Immunother. 52:328-337; Payne, (2003) Cancer Cell 3:207-212; Allen, (2002) Nat. Rev. Cancer 2:750-763; Pastan and Kreitman, (2002) Curr. Opin. Investig. Drugs 3:1089-1091; Senter and Springer, (2001) Adv. Drug Deliv. Rev. 53:247-264.

The antibodies, antibody fragments (e.g., antigen binding fragments) or functional equivalents of the present disclosure can also be conjugated to a radioactive isotope to generate cytotoxic radiopharmaceuticals, referred to as radioimmunoconjugates. Examples of radioactive isotopes that can be conjugated to antibodies for use diagnostically or therapeutically include, but are not limited to, iodine-131, indium-111, yttrium-90, and lutetium-177. Methods for preparing radioimmunoconjugates are established in the art. Examples of radioimmunoconjugates are commercially available, including Zevalin™ (DEC Pharmaceuticals) and Bexxar™M (Corixa Pharmaceuticals), and similar methods can be used to prepare radioimmunoconjugates using the antibodies disclosed herein. In certain aspects, the macrocyclic chelator is 1,4,7,10-tetraazacyclododecane-N,N′,N″,N″′-tetraacetic acid (DOTA) which can be attached to the antibody via a linker molecule. Such linker molecules are commonly known in the art and described in Denardo et al., (1998) Clin Cancer Res. 4(10):2483-90; Peterson et al., (1999) Bioconjug. Chem. 10(4):553-7; and Zimmerman et al., (1999) Nucl. Med. Biol. 26(8):943-50, each incorporated by reference in their entiretics.

The antibodies, antibody fragments (e.g., antigen binding fragments) or functional equivalents of the present disclosure can also conjugated to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the present disclosure provides fusion proteins comprising an antibody fragment (e.g., antigen binding fragment) described herein (e.g., a Fab fragment, Fd fragment, Fv fragment, F(ab)2 fragment, a VH domain, a VH CDR, a VL domain or a VL CDR) and a heterologous protein, polypeptide, or peptide.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the present disclosure or fragments thereof (e.g., antibodies or fragments thereof with higher affinities and lower dissociation rates). Sec, generally, U.S. Pat. Nos. 5,605,793, 5,811,238, 5,830,721, 5,834,252, and 5,837,458; Patten et al., (1997) Curr. Opinion Biotechnol. 8:724-33; Harayama, (1998) Trends Biotechnol. 16(2):76-82; Hansson et al., (1999) J. Mol. Biol. 287:265-76; and Lorenzo and Blasco, (1998) Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an antigen may be recombined with one or more components, motifs, sections, parts, domains, fragments, etc. of one or more heterologous molecules.

Moreover, the antibodies, antibody fragments (e.g., antigen binding fragments) or functional equivalents of the present disclosure can be conjugated to marker sequences, such as a peptide, to facilitate purification. In preferred aspects, the marker amino acid sequence is a hexa-histidine peptide, such as the tag provided in a pQE vector (QIAGEN, InC, 9259 Eton Avenue, Chatsworth, Calif., 91311), among others, many of which are commercially available. As described in Gentz et al., (1989) ProC Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “FLAG” tag (A. Einhauer et al., J. Biochem. Biophys. Methods 49: 455-465, 2001). As described in the present disclosure, antibodies or antigen binding fragments can also be conjugated to tumor-penetrating peptides in order to enhance their efficacy.

In other aspects, the antibodies, antibody fragments (e.g., antigen binding fragments) or functional equivalents of the present disclosure are conjugated to a diagnostic or detectable agent. Such immunoconjugates can be useful for monitoring or prognosing the onset, development, progression and/or severity of a disease or disorder as part of a clinical testing procedure, such as determining the efficacy of a particular therapy. Such diagnosis and detection can be accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, umbelliferone, fluorescein, fluorescein isothiocyanate, rhodamine, dichlorotriazinylamine fluorescein, dansyl chloride or phycoerythrin; luminescent materials, such as, but not limited to, luminol; bioluminescent materials, such as but not limited to, luciferase, luciferin, and acquorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Rc, ¹⁸⁸Re, ¹⁴²Pr, ¹⁰⁵Rh, ⁹⁷Ru , ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ⁶⁴Cu, ¹¹³Sn, and ¹¹⁷Sn; and positron emitting metals using various positron emission tomographies, and non-radioactive paramagnetic metal ions.

The antibodies, antibody fragments (e.g., antigen binding fragments) or functional equivalents of the present disclosure may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The ADCs may be prepared by any suitable method known in the art, such as those described in U.S. Pat. Nos. 7,811,572, 6,411,163, 7,368,565, and 8, 163,888, and US application publications 2011/0003969, 2011/0166319, 2012/0253021 and 2012/0259100. The entire teachings of these patents and patent application publications are herein incorporated by reference.

### One-Step Process

In one aspect, the conjugates of the present disclosure can be prepared by a one-step process. The process comprises combining the antibody, therapeutic compound and heterofunctional biolinker according to the disclosure in a substantially aqueous medium, optionally containing one or more co-solvents, at a suitable pH. In one aspect, the process comprises the step of contacting the antibody of the present disclosure with a therapeutic compound to form a first mixture comprising the antibody and the drug, and then contacting the first mixture comprising the antibody and the therapeutic compound with a heterofunctional biolinker according to the disclosure in a solution having a pH of about 4 to about 9 to provide a mixture comprising (i) the conjugate, (ii) free therapeutic compound, and (iii) reaction by-products.

In one aspect, the one-step process comprises contacting the antibody with the therapeutic compound and then the heterofunctional biolinker according to the disclosure in a solution having a pH of about 6 or greater (e.g., about 6 to about 9, about 6 to about 7, about 7 to about 9, about 7 to about 8.5, about 7.5 to about 8.5, about 7.5 to about 8.0, about 8.0 to about 9.0, or about 8.5 to about 9.0). For example, the process comprises contacting a cell-binding agent with the therapeutic compound (DM1 or DM4) and then the heterofunctional biolinker according to the disclosure in a solution having a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In another aspect, the process comprises contacting a cell-binding agent with the therapeutic compound and then the heterofunctional biolinker according to the disclosure in a solution having a pH of about 7.8 (e.g., a pH of 7.6 to 8.0 or a pH of 7.7 to 7.9).

The one-step process (i.e., contacting the antibody with the therapeutic compound and then the heterofunctional biolinker according to the disclosure can be carried out at any suitable temperature known in the art. For example, the one-step process can occur at about 20° C. or less (e.g., about −10° C. (provided that the solution is prevented from freezing, e.g., by the presence of organic solvent used to dissolve the cytotoxic agent and the heterofunctional biolinker) to about 20° C., about 0° C. to about 18° C., about 4° C. to about 16° C.), at room temperature (e.g., about 20° C. to about 30° C. or about 20° C. to about 25° C.), or at an elevated temperature (e.g., about 30° C. to about 37° C.). In one aspect, the one-step process occurs at a temperature of about 16° C. to about 24° C. (e.g., about 16° C., about 17° C., about 18° C., about 19° C., about 20° C., about 21° C., about 22° C., about 23° C., about 24° C., or about 25° C.). In another aspect, the one-step process is carried out at a temperature of about 15° C. or less (e.g., about −10° C. to about 15° C., or about 0° C. to about 15° C.). For example, the process comprises contacting the antibody with the therapeutic compound and then the heterofunctional biolinker according to the disclosure at a temperature of about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., about 0° C., about −1° C., about −2° C., about −3° C., about −4° C., about −5° C., about −6° C., about −7° C., about −8° C., about −9° C., or about −10° C., provided that the solution is prevented from freezing, e.g., by the presence of organic solvent(s) used to dissolve the heterofunctional biolinker according to the disclosure. In one aspect, the process comprises contacting the antibody with the therapeutic compound and then the heterofunctional biolinker according to the disclosure at a temperature of about −10° C. to about 15° C., about 0° C. to about 15° C., about 0° C. to about 10° C., about 0° C. to about 5° C., about 5° C. to about 15° C., about 10° C. to about 15° C., or about 5° C. to about 10° C. In another aspect, the process comprises contacting the antibody with the therapeutic compound and then the heterofunctional biolinker according to the disclosure at a temperature of about 10° C. (e.g., a temperature of 8° C. to 12° C. or a temperature of 9° C. to 11° C.).

In one aspect, the contacting described above is achieved by providing the antibody, then contacting the antibody with the therapeutic compound to form a first mixture comprising the antibody and the therapeutic compound, and then contacting the first mixture comprising the antibody and the therapeutic compound with the heterofunctional biolinker according to the disclosure. For example, in one aspect, the antibody is provided in a reaction vessel, the therapeutic compound is added to the reaction vessel (thereby contacting the antibody), and then the heterofunctional biolinker according to the disclosure is added to the mixture comprising the antibody and the therapeutic compound (thereby contacting the mixture comprising the antibody and the drug). In one aspect, the antibody is provided in a reaction vessel, and the therapeutic compound is added to the reaction vessel immediately following providing the antibody to the vessel. In another aspect, the antibody is provided in a reaction vessel, and the therapeutic compound is added to the reaction vessel after a time interval following providing the antibody to the vessel (e.g., about 5 minutes, about 10 minutes, about 20 minutes, about 30 minutes, about 40 minutes, about 50 minutes, about 1 hour, about 1 day or longer after providing the cell-binding agent to the space). The therapeutic compound can be added quickly (i.e., within a short time interval, such as about 5 minutes, about 10 minutes) or slowly (such as by using a pump).

The mixture comprising the antibody and the therapeutic compound can then be contacted with the heterofunctional biolinker according to the disclosure either immediately after contacting the antibody with the therapeutic compound or at some later point (e.g., about 5 minutes to about 8 hours or longer) after contacting the antibody with the therapeutic compound. For example, in one aspect, the heterofunctional biolinker according to the disclosure is added to the mixture comprising the antibody and the therapeutic compound immediately after the addition of the therapeutic compound to the reaction vessel comprising the antibody. Alternatively, the mixture comprising the antibody and the therapeutic compound can be contacted with the heterofunctional biolinker according to the disclosure at about 5 minutes, about 10 minutes, about 20 minutes, about 30 minutes, about 1 hour, about 2 hours, about 3 hours, about 4 hours, about 5 hours, about 6 hours, about 7 hours, about 8 hours, or longer after contacting the antibody with the therapeutic compound.

After the mixture comprising the antibody and the therapeutic compound is contacted with the heterofunctional biolinker according to the disclosure the reaction is allowed to proceed for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or longer (e.g., about 30 hours, about 35 hours, about 40 hours, about 45 hours, or about 48 hrs).

In one aspect, the one-step process further comprises a quenching step to quench any unreacted therapeutic compound and/or unreacted heterofunctional biolinker according to the disclosure. The quenching step is typically performed prior to purification of the conjugate. In one aspect, the mixture is quenched by contacting the mixture with a quenching reagent. As used herein, the “quenching reagent” refers to a reagent that reacts with the free therapeutic compound and/or heterofunctional biolinker according to the disclosure. In one aspect, maleimide or haloacetamide quenching reagents, such as 4-maleimidobutyric acid, 3-malcimidopropionic acid, N-ethylmaleimide, iodoacetamide, or iodoacetamidopropionic acid, can be used to ensure that any unreacted group (such as thiol) in the therapeutic compound is quenched. The quenching step can help prevent the dimerization of the therapeutic compound (e.g., DM1). The dimerized DM1 can be difficult to remove. Upon quenching with polar, charged thiol-quenching reagents (such as 4-maleimidobutyric acid or 3-maleimidopropionic acid), the excess, unreacted DM1 is converted into a polar, charged, water-soluble adduct that can be easily separated from the covalently-linked conjugate during the purification step. Quenching with non-polar and neutral thiol-quenching reagents can also be used. In one aspect, the mixture is quenched by contacting the mixture with a quenching reagent that reacts with the unreacted heterofunctional biolinker according to the disclosure. For example, nucleophiles can be added to the mixture in order to quench any unreacted heterofunctional biolinker. The nucleophile preferably is an amino group containing nucleophile, such as lysine, taurine and hydroxylamine.

In another aspect, the reaction (i.e., contacting the antibody with the therapeutic compound and then heterofunctional biolinker according to the disclosure) is allowed to proceed to completion prior to contacting the mixture with a quenching reagent. In this regard, the quenching reagent is added to the mixture about 1 hour to about 48 hours (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or about 25 hours to about 48 hours) after the mixture comprising the antibody and the therapeutic compound is contacted with the heterofunctional biolinker according to the disclosure.

Alternatively, the mixture is quenched by lowering the pH of the mixture to about 5.0 (e.g., 4.8, 4.9, 5.0, 5.1 or 5.2). In another aspect, the mixture is quenched by lowering the pH to less than 6.0, less than 5.5, less than 5.0, less than 4.8, less than 4.6, less than 4.4, less than 4.2, less than 4.0. Alternatively, the pH is lowered to about 4.0 (e.g., 3.8, 3.9, 4.0, 4.1 or 4.2) to about 6.0 (e.g., 5.8, 5.9, 6.0, 6.1 or 6.2), about 4.0 to about 5.0, about 4.5 (e.g., 4.3, 4.4, 4.5, 4.6 or 4.7) to about 5.0. In one aspect, the mixture is quenched by lowering the pH of the mixture to 4.8. In another aspect, the mixture is quenched by lowering the pH of the mixture to 5.5.

In one aspect, the one-step process further comprises a holding step to release the unstably bound biolinkers from the antibody. The holding step comprises holding the mixture prior to purification of the conjugate (e.g., after the reaction step, between the reaction step and the quenching step, or after the quenching step). For example, the process comprises (a) contacting the antibody with the therapeutic compound to form a mixture comprising the antibody and the therapeutic compound; and then contacting the mixture comprising the antibody and therapeutic compound with the heterofunctional biolinker according to the disclosure, in a solution having a pH of about 4 to about 9 to provide a mixture comprising (i) the conjugate, (ii) free therapeutic compound, and (iii) reaction by-products, (b) holding the mixture prepared in step (a) to release the unstably bound biolinkers from the cell-binding agent, and (c) purifying the mixture to provide a purified conjugate.

In another aspect, the process comprises (a) contacting the antibody with the therapeutic compound to form a mixture comprising the antibody and the therapeutic compound; and then contacting the mixture comprising the antibody and the therapeutic compound with the heterofunctional biolinker according to the disclosure, in a solution having a pH of about 4 to about 9 to provide a mixture comprising (i) the conjugate, (ii) free therapeutic compound, and (iii) reaction by-products, (b) quenching the mixture prepared in step (a) to quench any unreacted therapeutic compound and/or unreacted heterofunctional biolinker according to the disclosure, (c) holding the mixture prepared in step (b) to release the unstably bound biolinkers from the cell-binding agent, and (d) purifying the mixture to provide a purified conjugate.

Alternatively, the holding step can be performed after purification of the conjugate, followed by an additional purification step.

In another aspect, the reaction is allowed to proceed to completion prior to the holding step. In this regard, the holding step can be performed about 1 hour to about 48 hours (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or about 24 hours to about 48 hours) after the mixture comprising the antibody and the therapeutic compound is contacted with the heterofunctional biolinker according to the disclosure.

The holding step comprises maintaining the solution at a suitable temperature (e.g., about 0° C. to about 37° C.) for a suitable period of time (e.g., about 1 hour to about 1 week, about 1 hour to about 24 hours, about 1 hour to about 8 hours, or about 1 hour to about 4 hours) to release the unstably bound biolinkers from the antibody while not substantially releasing the stably bound biolinkers from the antibody. In one aspect, the holding step comprises maintaining the solution at about 20° C. or less (e.g., about 0° C. to about 18° C., about 4° C. to about 16° C.), at room temperature (e.g., about 20° C. to about 30° C. or about 20° C. to about 25° C.), or at an elevated temperature (e.g., about 30° C. to about 37° C.). In one aspect, the holding step comprises maintaining the solution at a temperature of about 16° C. to about 24° C. (e.g., about 15° C., about 16° C., about 17° C., about 18° C., about 19° C., about 20° C., about 21° C., about 22° C., about 23° C., about 24° C., or about 25° C.). In another aspect, the holding step comprises maintaining the solution at a temperature of about 2° C. to about 8° C. (e.g., about 0° C., about 1° C., about 2° C., about 3° C., about 4° C., about 5° C., about 6° C., about 7° C., about 8° C., about 9° C., or about 10° C.). In another aspect, the holding step comprises maintaining the solution at a temperature of about 37° C. (e.g., about 34° C., about 35° C., about 36° C., about 37° C., about 38° C., about 39° C., or about 40° C.).

The duration of the holding step depends on the temperature and the pH at which the holding step is performed. For example, the duration of the holding step can be substantially reduced by performing the holding step at elevated temperature, with the maximum temperature limited by the stability of the antibody drug conjugate. The holding step can comprise maintaining the solution for about 1 hour to about 1 day (e.g., about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, or about 24 hours), about 10 hours to about 24 hours, about 12 hours to about 24 hours, about 14 hours to about 24 hours, about 16 hours to about 24 hours, about 18 hours to about 24 hours, about 20 hours to about 24 hours, about 5 hours to about 1 week, about 20 hours to about 1 week, about 12 hours to about 1 week (e.g., about 12 hours, about 16 hours, about 20 hours, about 24 hours, about 2 days, about 3 days, about 4 days, about 5 days, about 6 days, or about 7 days), or about 1 day to about 1 week.

In one aspect, the holding step comprises maintaining the solution at a temperature of about 2° C. to about 8° C. for a period of at least about 12 hours for up to a week. In another aspect, the holding step comprises maintaining the solution at a temperature of about 2° C. to about 8° C. overnight (e.g., about 12 to about 24 hours, preferably about 20 hours).

The pH value for the holding step preferably is about 4 to about 10. In one aspect, the pH value for the holding step is about 4 or more, but less than about 6 (e.g., 4 to 5.9) or about 5 or more, but less than about 6 (e.g., 5 to 5.9). In another aspect, the pH values for the holding step range from about 6 to about 10 (e.g., about 6.5 to about 9, about 6 to about 8). For example, pH values for the holding step can be about 6, about 6.5, about 7, about 7.5, about 8, about 8.5, about 9, about 9.5, or about 10.

In other aspects, the holding step can comprise incubating the mixture at 25° C. at a pH of about 6-7.5 for about 12 hours to about 1 week, incubating the mixture at 4° C. at a pH of about 4.5-5.9 for about 5 hours to about 5 days, or incubating the mixture at 25° C. at a pH of about 4.5-5.9 for about 5 hours to about 1 day.

The one-step process can optionally include the addition of sucrose to the reaction step to increase solubility and recovery of the conjugates. In an embodiment, sucrose is added at a concentration of about 0.1% (w/v) to about 20% (w/v) (e.g., about 0.1% (w/v), 1% (w/v), 5% (w/v), 10% (w/v), 15% (w/v), or 20% (w/v)). Preferably, sucrose is added at a concentration of about 1% (w/v) to about 10% (w/v) (e.g., about 0.5% (w/v), about 1% (w/v), about 1.5% (w/v), about 2% (w/v), about 3% (w/v), about 4% (w/v), about 5% (w/v), about 6% (w/v), about 7% (w/v), about 8% (w/v), about 9% (w/v), about 10% (w/v), or about 11% (w/v)). In addition, the reaction step also can comprise the addition of a buffering agent. Any suitable buffering agent known in the art can be used. Suitable buffering agents include, for example, a citrate buffer, an acetate buffer, a succinate buffer, and a phosphate buffer. In one aspect, the buffering agent is selected from the group consisting of HEPPSO (N-(2-hydroxyethyl)piperazine-N′-(2-hydroxypropanesulfonic acid)), POPSO (piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

The one-step process can further comprise the step of purifying the mixture to provide purified conjugate. Any purification methods known in the art can be used to purify the conjugates of the present disclosure. In one aspect, the conjugates of the present disclosure use tangential flow filtration (TFF), non-adsorptive chromatography, adsorptive chromatography, adsorptive filtration, selective precipitation, or any other suitable purification process, as well as combinations thereof. In another aspect, prior to subjecting the conjugates to purification process described above, the conjugates are first filtered through one or more PVDF membranes. Alternatively, the conjugates are filtered through one or more PVDF membranes after subjecting the conjugates to the purification process described above. For example, in one aspect, the conjugates are filtered through one or more PVDF membranes and then purified using tangential flow filtration. Alternatively, the conjugates are purified using tangential flow filtration and then filtered through one or more PVDF membranes.

Any suitable TFF systems may be utilized for purification, including a Pellicon® type system, a Sartocon® Cassette system, and/or a Centrasette® type system.

Any suitable adsorptive chromatography resin may be utilized for purification. Preferred adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II), HA Ultrogel® hydroxyapatite, and ceramic fluoroapatite (CFT Type I and Type II). An example of a suitable HCIC resin is MEP Hypercel® resin. Examples of suitable HIC resins include Butyl-Sepharose, Hexyl-Sepaharose, Phenyl-Sepharose, and Octyl Sepharose resins, as well as Macro-Prep® Methyl and Macro-Prep® t-Butyl resins. Examples of suitable ion exchange resins include SP-Sepharose®, CM-Sepharose®, and Q-Sepharose® resins, and Unosphere® S resin. Examples of suitable mixed mode ion exchangers include Bakerbond® Abx resin. Examples of suitable IMAC resins include Chelating Sepharose® resin and Profinity® IMAC resin. Examples of suitable dye ligand resins include Blue Sepharose resin and Affi-gel Blue resin. Examples of suitable affinity resins include Protein A Sepharose resin and lectin affinity resins, e.g. Lentil Lectin Sepharose® resin, where the antibody bears appropriate lectin binding sites. Examples of suitable reversed phase resins include C4, C8, and C18 resins.

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

### Two-Step Process and One-Pot Process

In one aspect, the conjugates of the present disclosure can be prepared as described in the U.S. Pat. No. 7,811,572 and U.S. Patent Application Publication No. 2006/0182750. The process comprises the steps of (a) contacting the antibody of the present disclosure with the heterofunctional biolinker according to the disclosure to covalently attach the biolinker to the antibody and thereby prepare a first mixture comprising the antibody having the biolinker bound thereto; (b) optionally subjecting the first mixture to a purification process to prepare a purified first mixture of the antibody having the biolinker bound thereto; (c) conjugating the therapeutic compound to the antibody having the biolinker bound thereto in the first mixture by reacting the antibody having the biolinker bound thereto with the therapeutic compound in a solution having a pH of about 4 to about 9 to prepare a second mixture comprising (i) the conjugate, (ii) the therapeutic compound; and (iii) reaction by-products; and (d) subjecting the second mixture to a process to purify the conjugate from the other components of the second mixture. Alternatively, the purification step (b) can be omitted. Any purification methods described herein can be used for steps (b) and (d). In one embodiment, TFF is used for both steps (b) and (d). In another embodiment, TFF is used for step (b) and absorptive chromatography (e.g., CHT) is used for step (d).

### One-Step Reagent and In-Situ Process

In one aspect, the conjugates of the present disclosure can be prepared by conjugating pre-formed drug-linker compound to the antibody of the present disclosure, as described in U.S. Pat. No. 6,441,163 and U.S. Patent Application Publication Nos. 2011/0003969 and 2008/0145374, followed by a purification step. Any purification methods described herein can be used. The drug-linker compound is prepared by reacting the therapeutic compound with the heterofunctional biolinker according to the disclosure. The drug-linker compound is optionally subjected to purification before being conjugated to the antibody.

### Therapeutic and Diagnostic Uses of the ADCs

The ADCs of the present disclosure are useful in a variety of applications including, but not limited to, treatment of cancer and other diseases. In certain embodiments, the ADCs are useful for inhibiting tumor growth, inducing differentiation, reducing tumor volume, and/or reducing the tumorigenicity of a tumor. The methods of use can be in vitro, ex vivo, or in vivo methods.

In one embodiment, the ADCs are useful for detecting the presence of target in a biological sample. The term “detecting” as used herein encompasses quantitative or qualitative detection. In certain embodiments, a biological sample comprises a cell or tissue. In certain embodiments, such tissues include normal and/or cancerous tissues that express target at higher levels relative to other tissues.

In one embodiment, the present disclosure provides a method of detecting the presence of target in a biological sample. In certain embodiments, the method comprises contacting the biological sample with an antibody under conditions permissive for binding of the antibody to the antigen, and detecting whether a complex is formed between the antibody and the antigen.

Also included is a method of diagnosing a disorder associated with increased expression of target. In certain embodiments, the method comprises contacting a test cell with an antibody; determining the level of expression (either quantitatively or qualitatively) of target on the test cell by detecting binding of the antibody to the target antigen; and comparing the level of expression of target in the test cell with the level of expression of target in a control cell (e.g., a normal cell of the same tissue origin as the test cell or a cell that expresses target at levels comparable to such a normal cell), wherein a higher level of expression of target on the test cell as compared to the control cell indicates the presence of a disorder associated with increased expression of target. In certain embodiments, the test cell is obtained from an individual suspected of having a disorder associated with increased expression of target. In certain embodiments, the disorder is a cell proliferative disorder, such as a cancer or a tumor.

In certain embodiments, a method of diagnosis or detection, such as those described above, comprises detecting binding of an antibody to target expressed on the surface of a cell or in a membrane preparation obtained from a cell expressing target on its surface. An exemplary assay for detecting binding of an antibody to target expressed on the surface of a cell is a “FACS” assay.

Certain other methods can be used to detect binding of antibodies to target. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain embodiments, antibodies are labeled. In certain embodiments, antibodies are immobilized on an insoluble matrix. Any of the above embodiments of diagnosis or detection can be carried out using an immunoconjugate of the present disclosure in place of or in addition to an antibody.

In one embodiment, the disclosure provides for a method of treating, preventing or ameliorating a disease comprising administering the ADCs to a patient, thereby treating the disease. In certain embodiments, the disease treated with the ADCs is a cancer or other disease described herein. The present disclosure provides for methods of treating cancer comprising administering a therapeutically effective amount of the ADCs. In certain embodiments, the cancer is a solid cancer. In certain embodiments, the subject is a human.

In certain embodiments, the method of inhibiting tumor growth comprises administering to a subject a therapeutically effective amount of the ADCs. In certain embodiments, the subject is a human.

In certain embodiments, the tumor expresses the target to which the antibody binds. In certain embodiments, the tumor overexpresses the human target.

For the treatment of the disease, the appropriate dosage of the ADCs depend on various factors, such as the type of disease to be treated, the severity and course of the disease, the responsiveness of the disease, previous therapy, patient's clinical history, and so on. The antibody or agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual antibody, antibody fragment (e.g., antigen binding fragment), or ADCs. In certain embodiments, dosage is from 0.01 mg to 10 mg (e.g., 0.01 mg, 0.05 mg, 0.1 mg, 0.5 mg, 1 mg, 2 mg, 3 mg, 4 mg, 5 mg, 7 mg, 8 mg, 9 mg, or 10 mg) per kg of body weight, and can be given once or more daily, weekly, monthly or yearly. In certain embodiments, the antibody, antibody fragment (e.g., antigen binding fragment), or ADC of the present disclosure is given once every two weeks or once every three weeks. The treating physician can estimate repetition rates for dosing based on measured residence times and concentrations of the drug in bodily fluids or tissues.

### Combination Therapy

In certain instances, an ADC of the present disclosure is combined with other therapeutic agents, such as other anti-cancer agents, anti-allergic agents, anti-nausea agents (or anti-emetics), pain relievers, cytoprotective agents, and combinations thereof. In another embodiment, an ADC of the present disclosure is combined in a pharmaceutical combination formulation, or dosing regimen as combination therapy, with a second compound having anti-cancer properties. The second compound of the pharmaceutical combination formulation or dosing regimen can have complementary activities to the antibody or immunoconjugate of the combination such that they do not adversely affect each other. For example, an ADC the present disclosure can be administered in combination with, but not limited to, a chemotherapeutic agent, a tyrosine kinase inhibitor, for example, Imatinib, and other target pathway inhibitors. In another embodiment, the present disclosure provides a method of treating cancer by administering to a subject in need thereof an ADC in combination with one or more FGF downstream signaling pathway inhibitors, including but not limited to, MEK inhibitors, Braf inhibitors, PI3K/Akt inhibitors, SHP2 inhibitors, and also mTor. In yet another embodiment, the present disclosure provides a method of treating cancer by administering to a subject in need thereof an ADC in combination with one or more pro-apoptotics, including but not limited to, IAP inhibitors, Bc12 inhibitors, MC11 inhibitors, Trail agents, Chk inhibitors.

### Methods of Synthesizing a Heterofunctional Carbonyl Biolinker

Disclosed herein are methods for the preparation of a heterofunctional biolinker, the method comprising:
(a) converting a cyclohexane carboxylic acid according to the formula

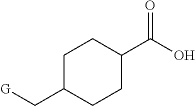

into a carboxylic acid alkyl halide, wherein G is any group that be converted into a leaving group (e.g., a halide);
(b) reacting the carboxylic acid alkyl halide with a protected hydroxylamine to yield a a protected alkoxyamine;
(c) converting a carboxyl group on the protected alkoxyamine into a reactive ester to yield a protected alkoxyamine reactive ester;
(d) deprotecting the protected alkoxyamine reactive ester;
(e) optionally, adding a spacer; and
(f) yielding a heterofunctional biolinker according to the structure:

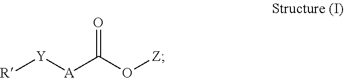

wherein R′ is —ONH₂ or absent; wherein A is a spacer; wherein Y is absent or an additional spacer; and wherein Z is a carboxylic acid activator that provides a reactive ester site.

In an embodiment, the spacer A and/or additional spacer Y comprises a substituted or unsubstituted, linear or branched C₁-C₂₀ hydrocarbon, polyethylene glycol (PEG), or a combination thereof.

In an embodiment, the spacer A is substituted or unsubstituted, linear or branched C₁-C₂₀ alkyl, alkenyl, alkynyl, cycloalkyl, aryl, cyclohexyl, or phenyl group. In a further embodiment, A is a cyclohexyl group.

In an embodiment, the carboxylic acid activator Z is a diimidyl group such as succinimidyl and phthalimidyl or substituted or unsubstituted, linear or branched C₁-C₂₀ alkyl, alkenyl, alkynyl, cycloalkyl, aryl, cyclohexyl, or benzyl group. In a further embodiment, Z is a succinimidyl group.

In a preferred embodiment, the cyclohexane carboxylic acid is a substituted cyclohexane carboxylic acid, more preferably comprising an —OH group, for example:

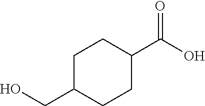 [SMILES: O=C(O)C1CCC(CO)CC1]

In a still further embodiment, the carboxylic acid is 4-(hydroxymethyl) cyclohexane carboxylic acid.

In a preferred embodiment, the carboxylic acid alkyl halide is a compound according to the structure:

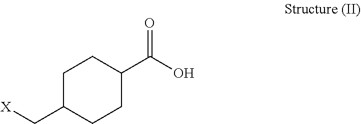

wherein X comprises a halide such as fluoride, chloride, bromide, or iodide. In an embodiment, the alkyl halide is an alkyl bromide, for example a compound according to the structure:

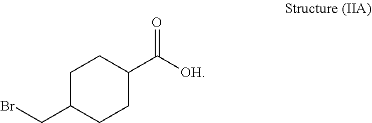 [SMILES: O=C(O)C1CCC(CBr)CC1]

In an embodiment, the protected hydroxylamine is an N-hydroxylamine. In a further embodiment, the hydroxylamine is a compound according to the structure:

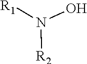

wherein R₁ is H and wherein R₂ is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

In an embodiment, the protected hydroxylamine is phenyl hydroxylamine, tolyl hydroxylamine, 2-iodophenyl hydroxylamine, N-benzyl hydroxylamine, N-methoxycarbonyl hydroxylamine, N-ethoxycarbonyl hydroxylamine, N-Boc hydroxylamine, N-CBz hydroxylamine, N-Fmoc hydroxylamine, N-hydroxysuccinimide, N-hydroxyphthalimide or a combination thereof. Structural examples of protected hydroxylamines include:

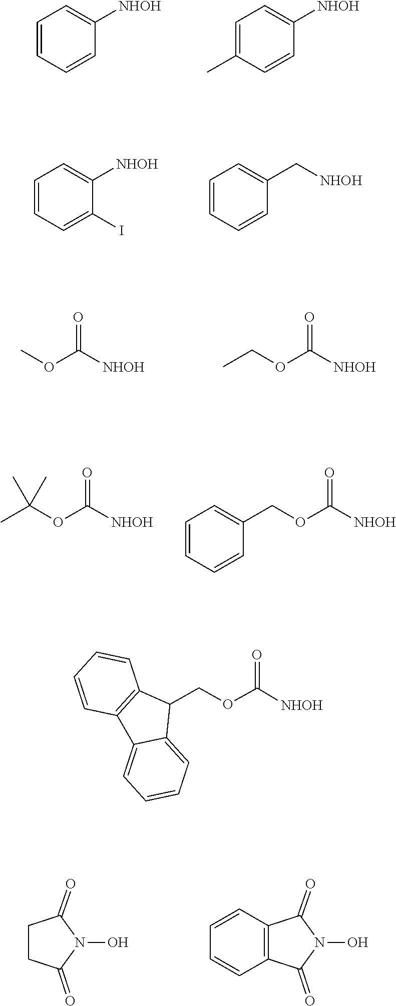

In an embodiment, the protected alkoxyamine is a compound according to the structure:

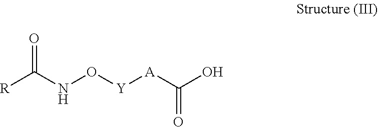

wherein R is independently selected from H, —OC(CH₃)₃, —OCH₂R where R is H, methyl, phenyl or fluorenyl, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof and the additional spacer Y is independepently selected from a substituted or unsubstituted, linear or branched C₁-C₂₀ hydrocarbon, polyethylene glycol (PEG), or a combination thereof and the spacer A is independently selected from substituted or unsubstituted, linear or branched C₁-C₂₀ alkyl, alkenyl, alkynyl, cycloalkyl, aryl, cyclohexyl, or phenyl group or a combination thereof.

In a preferred embodiment, R is —OC(CH₃)₃, and Y is a methylene (CH₂) and A is a substituted cyclohexane, particularly a 1,4-disubstituted cyclohexane carboxylic acid, preferably according to the structure:

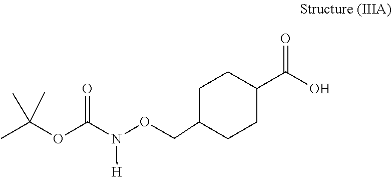 [SMILES: CC(C)(C)OC(=O)NOCC1CCC(C(=O)O)CC1]

In an embodiment, the protected alkoxyamine reactive ester is a compound according to the structure:

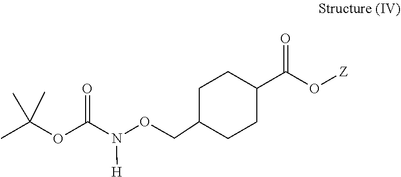

wherein Z is a carboxylic acid activator that provides a reactive ester site.

More preferably, the protected alkoxyamine reactive ester is a compound according to the structure:

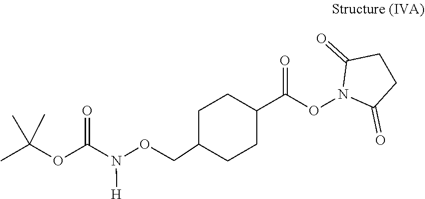 [SMILES: CC(C)(C)OC(=O)NOCC1CCC(C(=O)ON2C(=O)CCC2=O)CC1]

### Methods of Synthesizing a Succinimidyl 4-(aminooxymethyl)cyclohexane-1-carboxylate (SAOCC) Biolinker

Disclosed herein are versatile methods of synthesizing a SAOCC biolinker according to the structure:

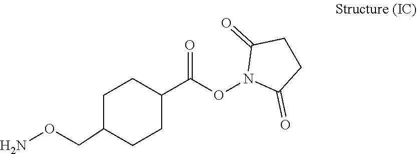 [SMILES: NOCC1CCC(C(=O)ON2C(=O)CCC2=O)CC1]

The compound according to Structure (IC) can be generated through a method comprising:
(1) adding triphenylphosphine to a stirred solution of 4-(hydroxymethyl)cyclohexane carboxylic acid and carbon tetrabromide in CH₂Cl₂ at 0° C. Preferably, to further dissolve the reagents, additional CH₂Cl₂ is added as needed and the reaction is allowed to stir for 48 hours at room temperature. When complete by TLC (25% EtOAc and 0.01% acetic acid in heptane, followed by staining with p-anisaldehyde), the reaction mixture is removed from stirring and filtered through a pad of silica gel to afford Structure (IIA) as an oil (87% yield) as shown below:

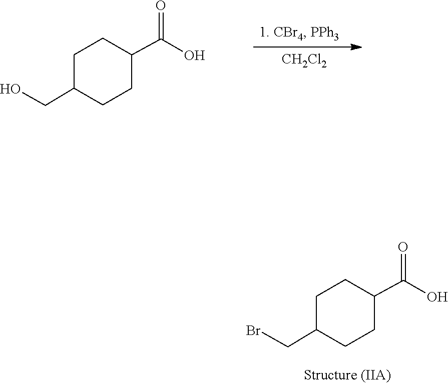 [SMILES: O=C(O)C1CCC(CO)CC1 ; "CH2Cl2" ; O=C(O)C1CCC(CBr)CC1]

(2) adding N-Boc-hydroxylamine (BocNHOH) to a solution of Structure (IIA) in Et₃N. The resultant reaction mixture is then preferably heated to 50° C. for 3 hours. When complete by TLC (25% EtOAc in Heptane, followed by staining with p-anisaldehyde), the reaction mixture was concentrated in vacuo to remove as much Et₃N as possible. The resulting residue is preferably dissolved in Et2O and washed with saturated NH₄Cl (aq) followed by H₂O and saturated NaCl (aq). The organic layer is preferably dried over MgSO₄ and the solvent is removed in vacuo. The residue is then preferably dissolved in CH₂Cl₂ and washed with cold H₂O (2×) followed by saturated NaCl (aq). The organic layer is then preferably dried over MgSO₄ and the solvent is removed in vacuo to afford Structure (IIIA) as an oil (50% yield) as shown below:

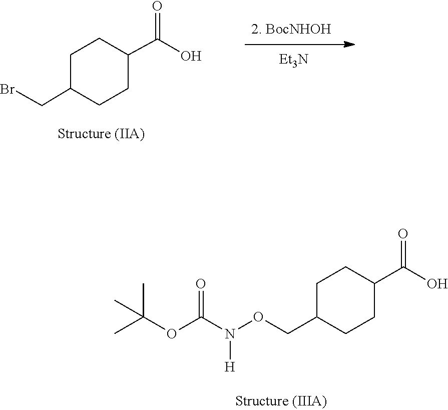 [SMILES: "Et3N" ; O=C(O)C1CCC(CBr)CC1 ; CC(C)(C)OC(=O)NOCC1CCC(C(=O)O)CC1]

(3) adding N-hydroxysuccinimide and DCC to a solution of Structure (IIIA) in CH3CN. The resultant reaction mixture is preferably stirred at room temperature for 24 hours. When complete by TLC (25% EtOAc in Heptane, followed by staining with p-anisaldehyde), the reaction mixture is preferably cooled to −78° C. and filtered through celite. The filtrate is then preferably concentrated in vacuo to afford Structure (IVA) as an oil (99% yield) as shown below:

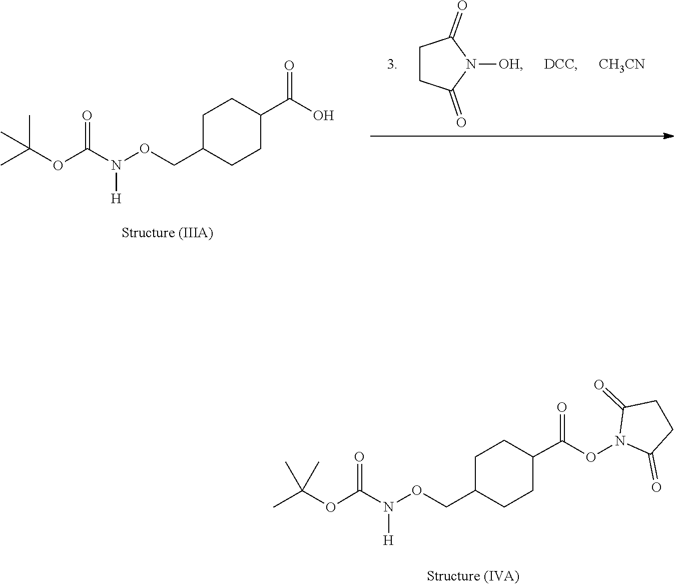

(4) adding TFA to a solution of Structure (IVA) in CH₂Cl₂. The resultant reaction mixture is preferably stirred at room temperature for 2 hours. When complete by TLC (25% EtOAc in Heptane, followed by staining with p-anisaldehyde), the reaction mixture is then preferably concentrated in vacuo to afford Structure (IC), the SAOCC (Succinimidyl 4-(aminooxymethyl)cyclohexane-1-carboxylate) Biolinker, as an oil (99% yield) as shown below:

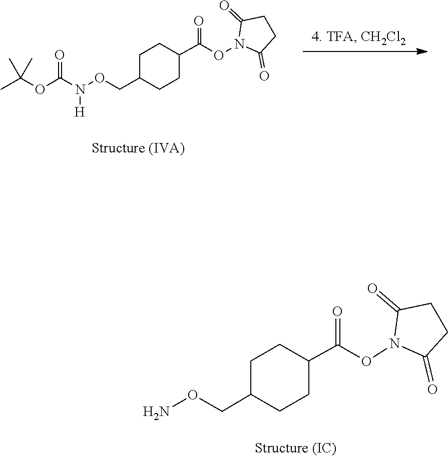 [SMILES: CC(C)(C)OC(=O)NOCC1CCC(C(=O)ON2C(=O)CCC2=O)CC1 ; NOCC1CCC(C(=O)ON2C(=O)CCC2=O)CC1]

### Methods of Detecting a Target

As described herein, biolinkers having a high affinity and specificity for aldehyde- and ketone-containing biomarkers are extremely useful research tools and would enable a number of new or improved diagnostic and therapeutic applications. However, achieving high affinity and high specificity carbohydrate recognition has historically been extremely challenging. In molecular recognition, antibodies are typically considered the gold standard. However, in the case of carbohydrates, raising antibodies is often difficult or impossible. More often than not, low-affinity IgM antibodies are obtained. Attempts to solve this problem involve costly and challenging methods, such as the generation and use of artificial lectins. Beneficially, the heterofunctional biolinkers described herein can be synthesized in four steps from commercially available reagents, thereby enabling cost-effective and highly accurate detection of a target.

In some embodiments, the methods of detecting a target involve covalently bonding a biomarker to the biolinker, in particular the alkoxyamine (RONH₂) end of the biolinker. In the instant case, the reaction between the alkoxyamine on the heterofunctional biolinker and the aldehyde or ketone carbonyl moiety of the biomarker occurs as follows:

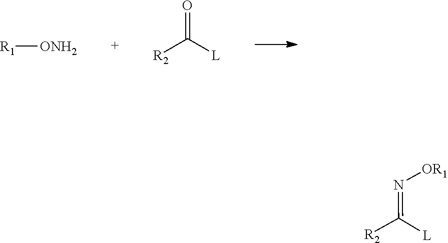

wherein R₁ is the remainder of the heterofunctional biolinker and R₂ is H or an alkyl group; and wherein L is a biomarker as described herein, such as a carbohydrate, cofactor, nucleic acid peptide, protein, glycoprotein, or another glycosylated molecule that contains the target functional group, e.g., an aldehyde of a carbohydrate-sugar group.

An example method for labeling a biomarker on a target is discussed. In one embodiment, a sample, such as blood or saliva, is obtained. The sample is suspected of including at least one target biomarker and optionally an analyte. The sample may be collected by any suitable device, system, or method of sample collection, such as those described in one or more of the following U.S. patents and published applications, each of which is hereby incorporated by reference in its entirety: U.S. Pat. Nos. 7,074,577; 7,220,593; 7,329,534; 7,358,095; 7,629,176; 7,915,029; 7,919,049; 8,012,742; 9,039,999; 9,217,697; 9,492,819; 9,513,291; 9,533,303; 9,539,570; 9,541,481; 9,625,360; 2014/0161688; 2017/0014819; 2017/0059552; 2017/0074759. Suitable devices, systems, and methods for sample retrieval, isolation, or selection can include those described in one or more of the following U.S. patents and published applications, each of which is hereby incorporated by reference in its entirety: U.S. Pat. Nos. 9,222,953; 9,227,188; 9,440,234; 9,519,002; 9,810,605; 2017/0219463; 2017/0276575.

Once collected, the sample is then contacted with an amount of the heterofunctional biolinker as described herein sufficient to permit the heterofunctional biolinker covalently bind with the biomarker in the sample. It will be appreciated that the heterofunctional biolinker will have already been synthesized using a label suitable for bonding with and detecting the target.

The reacted mixture of the biolinker and sample is then dispensed onto or into at least one analytical device. The analytical device may comprise, for example, a microscope slide, a positively charged microscope slide, a negatively charged microscope slide, a coated microscope slide, a porous slide, a micro-well slide, a well plate, a coverslip, a cell microarray, or the like. In an embodiment, the analytical device is a microscale device. In a preferred embodiment, the analytical device is a point-of-care testing device. The analytical device may further incorporate other features permitting complex functions, for example, the integration of nanostructures, electrodes, wax-printed butyrylcholinesterase (BchE) paper sensors, or surface functionalization through any suitable method, such as thin film deposition, plasma etching, lamination, or the like. Further discussion of surface functionalization of microfluidic devices can be found, for example, in Eichler, M., Klages, C-P., & Lachmann, K. (2016). Surface Functionalization of Microfluidic Devices. Microsystems for Pharmatechnology, 59-97. Doi: 10.1007/978-3-319-26920-7_3, which is herein incorporated by reference in its entirety. Suitable types of analytical devices include a microscale device, optical immunosensor, spectrometer, a benchtop analytical device such as a blood gas analyzer, infrared sensor, meter, microfluid analytical device, such as a microfluidic chip, or a microfluidic paper-based analytical device, test strip, or a combination thereof. One or more analytical devices may operate in tandem, e.g., a paper-based test strip comprising a biological component such as an enzyme and an electrode or other feature on a paper-based substrate that interfaces with a meter comprising a sensor and/or an electrode.

The solid-state storage system may be used with or incorporated into one or more microfluidic devices such as microfluidic chips or paper-based microfluidic devices. Microfluidic chips comprise a pattern of microchannels engraved or etched into a material such as glass, silicon, paper, or a polymer like PDMS. The diameter of the microchannels typically ranges between about 100 nm and about 100 microns. This network of microchannels is connected to the macro-environment through one or more entry points on the chip. Fluid enters the chip through one or more flow control devices in order to achieve a desired effect, such as mixing, pumping, reacting, sorting, or controlling the biochemical environment. Additionally, low-cost paper-based microfluidic analytical devices are a useful type of lab-on-chip (LOC) platform that permits on and off-site analysis. In particular, microfluidic paper-based analytical devices (μPADs) provide rapid operation and precise interpretations while still being cost-effective. Another benefit is that μPADs are highly compact, portable, easy to use, and do not require additional sophisticated equipment to operate. Overall, microfluidic devices provide multi-process functionality compatible with a wide variety of biological systems. For example, the solid-state storage system may be used in conjunction with microfluidic PCR, qPCR, RT-PCR, qRT-PCR, pH control, drug administration, cell analysis, or diagnostics.

In an embodiment, the analytical device comprises a paper-based microfluidic device. In some embodiments, the paper-based microfluidic device comprises a porous substrate preferably comprising paper, an input channel, a reaction chamber, one or more microfluidic channels, and a non-porous barrier that defines one or more boundaries for the reaction chamber and the one or more microfluidic channels, wherein the reaction chamber is fluidly connected to the input chamber by the one or more microfluidic channels, and wherein the surface of the microfluidic device is laminated except for the reaction chamber. In an embodiment, the non-porous barrier comprises wax. In an embodiment, the paper comprises nitrocellulose acetate, cellulose acetate, cellulosic paper, filter paper, tissue paper, writing paper, paper towel, cloth, or porous polymer film. The paper-based microfluidic device may comprise one or more input channels and one or more reaction chambers. Additionally, the surface of the microfluidic device, the reaction chamber, or the one or more channels may be functionalized. In some embodiments, the paper-based microfluidic device further comprises one or more conductive metals (e.g., Sn, Zn, Au, Ag, Ni, Pt. Pd, Al, In, or Cu), one or more polymers (e.g., a photoresist or a curable polymer), one or more insulating materials, or a combination thereof.

In one embodiment, the sample may be suspended in an attachment solution in a vessel or container prior to being dispensed onto the analytical device. For example, an attachment solution, buffer, reagent, or aqueous solution may be added to or mixed with the sample. The suspended sample is then dispensed onto or into the analytical device by a dispenser, such as a pipet or repeating pipet. Alternatively, in an embodiment, the sample need not be suspended.

In one embodiment, the sample is spread across the analytical device. In one embodiment, the sample is spread across the analytical device by a spreader, such as a squeegee, a pipet tip, a blade, or a two-piece spreader including a blade and a base. In one embodiment, the sample is spread across the analytical device by centrifuging, wetting, or nutating the analytical device. In another embodiment, when the sample is suspended, the sample is cured to adhere the suspended sample to the analytical device. In an alternative embodiment, the suspended sample is dispensed onto the analytical device and cured without being spread across the analytical device. Curing occurs in air, such as at room temperature; in an environmentally-controlled chamber, such as at 37° C.; or the like. Furthermore, the sample can undergo an additional fixation step, such as in treatment with formalin or any appropriate fixative, after the curing step has been completed.

Following deposition in or on the analytical device, the biomarker is detected and/or quantified. Detection and quantification can occur through any method suitable for the type of label used. For example, quantification can be done with a flow cytometer or a microscope, such as a fluorescent microscope, a scanner, or any other appropriate imaging system or modality.

In one embodiment, quantification can be performed via photographic images using visible light, infrared light, or ultraviolet light. In an alternative embodiment, where the label is fluoresced, quantification can be performed by exposing the sample to a light source, detecting an excitation output from the sample, and quantifying the excitation output and the concentration of the biomarker (and by extension the target).

In an embodiment, quantification is conducted through the use of a sample reader, particularly a portable sample reader comprising: an optical system comprising a light source, a filter, and one or more of a photosensor, a detector, or an imager; and a housing unit configured to receive one or more sample containers. In an embodiment, the light source comprises a light emitting diode (LED); and/or the filter comprises a band-pass filter, a long-pass filter, a short-pass filter, or a combination thereof. According to an embodiment, the photosensor comprises a photosensor chip; the detector comprises a photodetector; and/or the imager comprises a compact camera, bridge camera, DSLR camera, high-resolution microscopy camera, CMOS camera, sCMOS camera, CCD-chip camera, USB camera, USB camera controller board, or a combination thereof.

In one embodiment, multiple rounds of labeling can be performed for cyclic labeling purposes. For example, a target may be labeled with a first label by contacting a sample believed to contain the target with the heterofunctional biolinkers described herein. Following contacting, the sample is optionally imaged, and optionally, the signal provided by the first label is reduced or eliminated, such as with heat, light, chemicals, enzymes, molecules having a higher affinity to remove or inactivate the reporter, such as by denaturing, degrading, cleaving, removing, or modification. When heat is used, any suitable degree of heat may be applied, such as 120° C., including at least 30° C., 40° C., 50° C., 60° C., 70° C., 80° C., 90° C., 95° C., 100° C., 105° C., 110° C., 115° C., or 119° C. Subsequently, the target is labeled with a second label by contacting the sample with a heterofunctional biolinker comprising the same or a different reporter. This process may be repeated until a desired number of biomarkers have been labeled and/or a desired number of rounds have been performed. Imaging may be performed after multiple rounds of contacting the sample with biolinkers comprising several different labels.

### Clinical and Therapeutic Uses of Carbonyl-Containing Biomarkers

Carbonyl-containing biomarkers and particularly carbohydrate biomarkers play a significant role in a wide range of biological and pathological processes. In particular, carbohydrates have many versatile functions in a large number of biological processes such as fertilization, pregnancy progression, signal transduction, protein functioning and regulation, cell-cell communications, stem cell differentiation, and embryonic development. Recent advances in glycomics and glycoproteomics have contributed immensely to the understanding of glycan-associated aberrations in diseased states. For example, most cancer types show aberrant cancer-specific glycosylations, reflecting the abnormal expression of enzymes that are implicated in glycan biosynthesis such as glycosyltransferases and glycosidases. Consequently, cancer cells produce glycoproteins and glycolipids with modified glycan structures as opposed to their normal counterparts. Such anomalous glycan modifications and altered glycosyltransferase and glycosidase levels provide a compelling platform for their exploitation in the improvement and development of diagnostics and therapeutics. For instance, in the case of cancer, most of the Food and Drug Administration (FDA)-approved biomarkers are carbohydrate-dependent.

Further, carbohydrate biomarkers are involved in, and useful for the detection of, a variety of pathogens. For example, in the case of the human influenza virus, the viral infection process utilizes sialic acid for binding to hemagglutinin. After infection, the development of mature viruses from infected cells involves the cleavage of sialic acid by neuraminidase in order for the virus to detach. In the case of the human immunodeficiency virus (HIV), a critical protein, gp120, is glycosylated with polymannose. Infection of cells by HIV-1 requires the fusion of the viral membrane with the cellular membrane. This fusion is mediated by gp 120 and gp41 along with cell surface receptors on the target cells. Carbohydrates also play important roles in bacterial pathogenicity. For gram-negative bacteria, lipopolysaccharides (LPS) stimulate the immune system in an attempt to clear the bacteria. The lipid A component of LPS is primarily responsible for this inflammatory response. As the infection proceeds, the presence of a large amount of LPS can result in an overproduction of inflammatory mediators that result in damage to tissues, septic shock, organ failure, and death. In addition to functioning as toxins, carbohydrates can also be the target for bacterial recognition. For example, many pulmonary pathogenic bacteria bind specifically to the carbohydrate sequence GalNAc beta 1-4Gal found in some glycolipids.

Beyond pathogens, carbohydrates are also involved in mediating inflammatory processes, immune responses, and cardiovascular disease. For example, glycated hemoglobin (GlcHb) level is a strong indicator of cardiovascular disease (CVD) risk in patients, particularly diabetic patients.

Accordingly, biomarkers containing aldehydes and ketones are useful for a wide variety of diagnostic and therapeutic applications, for example in the detection of cancer stem cells; cancers such as breast, colon, lung, bladder, cervix, ovary, stomach, and prostate cancer; Down syndrome; infectious diseases; inflammatory responses; as well as pathological conditions including cancer metastasis, inflammation, bacterial and viral infections, immune responses, and cardiovascular malfunctions.

### Controlled Release of Substances

The biolinkers described herein can also be used as part of the controlled release of substances having a carbonyl group. There are several situations where it is desirable to control the release of a ketone-or aldehyde-containing group. For example, it may be desirable to control the release of a ketone-or aldehyde-based drug or therapeutic to a cell population or target site (e.g., a tissue or an organ). It is also desirable to deliver biologically active compounds to selected cells in a heterogeneous cell population. For example, in treating diseased or infected cells such as virus-infected cells or transformed or malignant cells, it is desirable to deliver cytotoxins to the diseased or malignant cells but not to normal cells.

Disclosed herein are methods of delivering a substance to a target comprising providing a heterofunctional biolinker to a media (such as a liquid or aerosolized media); and exposing the target to the heterofunctional biolinker, wherein the substance is conjugated to the heterofunctional biolinker.

In an embodiment, the substance is a compound used for the treatment of a disease such as HIV, osteoarthritis, rheumatoid arthritis, Parkinson's disease, cancer (such as melanoma, multiple myeloma, ovarian cancer, pancreatic cancer), tumors, Type II diabetes, gastroenteritis, ulcers, asthma, chronic obstructive pulmonary disease (COPD), migraines, or a combination thereof. In an embodiment, the substance is a compound for the treatment of a psychiatric disease such as a sleep disorder, a psychomotor dysfunction, bipolar disorder, seasonal affective disorder (SAD), major depressive disorder (MDD), schizophrenia, a chronic psychosis, or a combination thereof.

Examples of substances having a ketone group include, but are not limited to, tolcapone, methadone, nabumetone, haloperidol, bromfenac, fenofibrate, buproprion, camphor, acetophenone, melperone, ebastine, chlorthalidone, nitisinone, droperidol, dyclonine, propafenone, levacetylmethadol, oxybenzone, methadyl acetate, dipipanone, alphacetylmethadol, acetoacetic acid, benzophenone, cyclohexanone, thiocamphor, hexafluoroacetone hydrate, isoliquiritigenin, 4-hydroxybutan-2-one, thenoyltrifluoroacetone, imexon, clafibranor, sofalcone, tolperisone, R-95845, phloretin, p-benzoyl-L-phenylalanine, eprazinone, eperisone, pipamperone, avobenzone, exametazime, sulisobenzone, enzacamene, dioxybenzone, diethylamino hydroxybenzoyl hexyl benzoate, azaperone, tiletamine, normethadone, radafaxine, bromperidol, apocynin, benperidol, oxyfedrine, trifluperidol, moperone, morclofone, desaspidin, fluanisone, etafenone, fenofibric acid, benzoin, dihydroxymethoxychalcone, nebicapone, dextromethadone, methcathinone, xanthohumol, pitofenone, ketocaine, azemiglitazone, paconol, spiperone, or a combination thereof.

Examples of substances having an aldehyde group include, but are not limited to, phenylacetaldehyde, malonaldehyde, phosphonoacetaldehyde, p-hydroxybenzaldehyde, formaldehyde, protocatechualdehyde, tucaresol, cinnamaldehyde, glutaral, pyruvaldehyde, chlorhexadol, voxelotor, furfural, or a combination thereof.

### Testing Kit

In an embodiment, the heterofunctional biolinkers described herein may be provided as part of a kit for collecting and testing samples. The kit comprises, for example, one or more sample containers comprising the heterofunctional biolinkers described herein, a sample reader configured to receive the one or more sample containers, a sample collector such as a lancet, a blood collection capillary, a saliva collection cup, a vial, a collection cup, a swab, a nasopharyngeal swab, or a combination thereof, a power source, instructions for using the testing kit, a carrier for storage and transport of each of the components of the kit, or any combination thereof.

The raw sample comprises a basic substance in its natural, modified, or semi-processed state, wherein the sample is generally not yet fully processed or prepared. The raw samples generally contain wholly or a high quantity of intact cells, i.e., cells that have not yet been intentionally lysed. Although some cells in a raw sample may be ruptured due to natural causes or the state of the sample upon collection, a raw sample according to the present application does not contain cells intentionally ruptured, or otherwise processed or prepared.

In an embodiment, the raw sample comprises a sample from an individual including, but not limited to, blood, saliva, urine, spinal fluid, nasal discharge, or a combination thereof. In an alternative embodiment, the raw sample comprises water (e.g., wastewater, water from a body of water, tap water, or the like), soil, or an organic sample.

In an embodiment, the testing kit comprising the heterofunctional biolinkers is integrated into a sample-to-answer platform. The term “sample-to-answer” refers to a platform that is fully integrated such that it can perform an analysis of complex biological samples on a largely or fully automated basis. Sample-to-answer platforms can be beneficially used at the point of care, i.e., near or at the side of a patient or on-site (e.g., at an environmental testing site).

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1. Synthesis of a SAOCC Biolinker

The following are the experimental descriptions of the four synthetic steps that were used to synthesize a gram quantity of SAOCC:

### Step 1

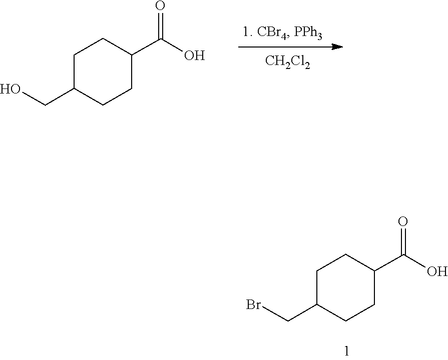 [SMILES: O=C(O)C1CCC(CO)CC1 ; "CH2Cl2" ; O=C(O)C1CCC(CBr)CC1]

To a stirred solution of 4-(hydroxymethyl) cyclohexane carboxylic acid (0.5 g, 3.2 mmol, 1 eq.) and carbon tetrabromide (1.89 g, 5.7 mmol, 1.8 eq.) in CH₂Cl₂ (5 mL) at 0° C., triphenylphosphine (1.66 g, 6.3 mmol, 2 eq.) was added. To further dissolve the reaction, additional CH₂Cl₂ (7 mL) was added and the reaction was allowed to stir for 48 hours at room temperature. When complete by TLC (25% EtOAc and 0.01% acetic acid in Heptane, followed by staining with p-anisaldehyde), the reaction mixture was removed from stirring and filtered through a pad of silica gel. Removal of the solvents in vacuo afforded 1 as an oil (87% yield). The product was analyzed by NMR and IR.

In developing step 1, it is noted that the use of a halide leaving group was surprisingly more successful than other commonly used leaving groups. Initially, the installation of the tosylate (OTs) instead of the bromide (Br) leaving group was attempted. 4-(hydroxymethyl) cyclohexane carboxylic acid (1 eq.) was allowed to react with p-toluenesulfonylchloride (1.1 eq.) in the presence of triethylamine (1.2 eq.) and N,N-dimethylaminopyridine (0.1 eq.) in CH₂Cl₂ at room temperature. Even after a week, there was little product. Tosylate was abandoned for bromide as the leaving group.

Further, although the relative amounts of carbon tetrabromide and triphylphosphine can be modified, the described ratios provide an improved reaction rate and yield. Different amounts of carbon tetrabromide and triphenylphosphine were evaluated, but the reaction time and/or yield suffered. For example, when 1.1 eq. CBr₄ was used with 1.2 eq. PPh₃, the reaction took longer than 48 hours to reach completion. When 2.2 eq. CBr₄ was used with 2.4 eq. PPh₃, the reaction yield decreased to 73% presumably due to more involved purification.

### Step 2

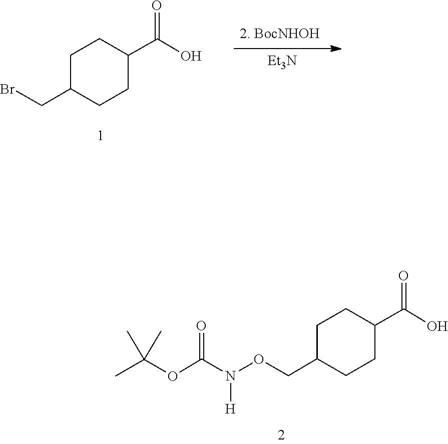 [SMILES: "Et3N" ; O=C(O)C1CCC(CBr)CC1 ; CC(C)(C)OC(=O)NOCC1CCC(C(=O)O)CC1]

To a solution of 1 (1.0 g, 4.5 mmol, 1 eq.) in Et₃N (50 mL) was added BocNHOH (0.67 g, 5.0 mmol, 1.1 eq.). The resultant reaction mixture was heated to 50° C. for 3 h. When complete by TLC (25% EtOAc in Heptane, followed by staining with p-anisaldehyde), the reaction mixture was concentrated in vacuo to remove as much Et₃N as possible. The resulting residue was dissolved in Et₂O and washed with saturated NH₄Cl (aq) followed by H₂O and saturated NaCl (aq). The organic layer was dried over MgSO₄ and the solvent was removed in vacuo. The residue was dissolved in CH₂Cl₂ and washed with cold H₂O (2×) followed by saturated NaCl (aq). The organic layer was dried over MgSO₄ and the solvent was removed in vacuo to afford 2 as an oil (50% yield). The product was analyzed by NMR and IR.

For this step, a triethylamine (Et₃N) base is preferred. Although potassium or cesium carbonate can be used as a base, along with tetrahydrofuran (THF) or N,N-dimethylformamide (DMF) as the solvent, these reaction conditions provided lower yields and more impurities.

Further, the combination of CH₂Cl₂ and diethyl ether (Et₂O), used at different stages is preferred. Although dichloromethane (CH₂Cl₂) may be used, it may result in isolated products with residual triethylammonium bromide (Et₃NHBr) salts. Beneficially, diethyl ether does not dissolve the triethylammonium bromide (Et₃NHBr) salts, which end up dissolving in the aqueous layer. However, diethyl ether dissolves the desired product 2 effectively.

When the entire work up is performed with dichloromethane (CH₂Cl₂), the isolated products is often contaminated with residual triethylammonium bromide (Et₃NHBr) salts. In order to fix this, diethyl ether (Et₂O), a solvent does not dissolve these salts and push them out of the organic layer into the aqueous layer, was employed. Unfortunately, Et₂O dissolved unreacted BocNHOH very thoroughly, resulting in some contamination of the isolated product with BocNHOH. In contrast, CH₂Cl₂ did not dissolve BocNHOH effectively. The use of CH₂Cl₂ allowed the washing away of any unreacted BocNHOH with H₂O while the desired product stayed in the CH₂Cl₂ layer. Thus, step 2 incorporates both Et₂O and CH₂Cl₂ to isolate and purify the desired product and eliminated the need for any further purification.

### Step 3

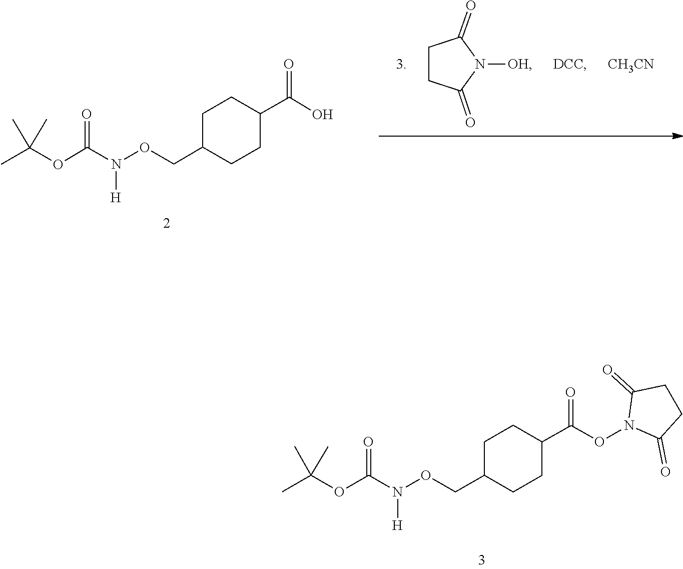

To a solution of 2 (1.0 g, 3.7 mmol, 1.0 eq.) in CH₃CN (20 mL) were added N-hydroxysuccinimide (0.43 g, 3.7 mmol, 1.0 eq.) and DCC (0.76 g, 3.7 mmol, 1.0 eq.). The resultant reaction mixture was stirred at room temperature for 24 h. When complete by TLC (25% EtOAc in Heptane, followed by staining with p-anisaldehyde), the reaction mixture was cooled to −78° C. and filtered through celite. The filtrate was concentrated in vacuo to afford 3 as an oil (99% yield). The product was analyzed by NMR and IR.

A full purification step is beneficially avoided in step three through cooling and filtration. Initial use of an ice bath to precipitate out the solids (e.g., dicyclohexylurea by-product) and filtration through a fritted glass funnel resulted in acceptable removal of solids, but some dicyclohexylurea was still found in the isolated product, presumably due to insufficient precipitation of dicyclohexylurea from CH₃CN as dicyclohexylurea has some solubility in CH₃CN. It was found that cooling the reaction mixture to −78° C. using acetone and dry ice bath and filtration using a fritted glass funnel lead to less dicyclohexylurea in the isolated product solids using this method, although a small amount still remained. Thus, the steps of cooling to −78° C. and filtering through fritted glass funnel were repeated multiple times to get rid of all of dicyclohexylurea by-products. Ultimately, it was determined that cooling to −78° C. and filtering through celite removed all of the dicyclohexylurea by-products, thereby eliminating a need for further purification.

### Step 4

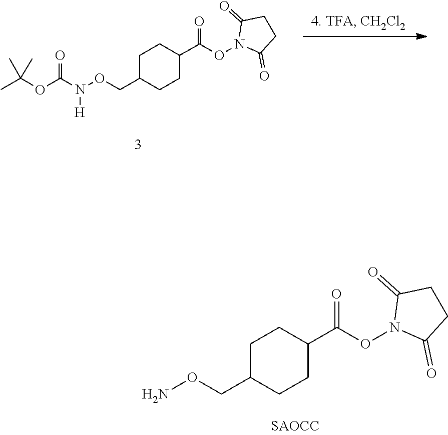 [SMILES: CC(C)(C)OC(=O)NOCC1CCC(C(=O)ON2C(=O)CCC2=O)CC1 ; NOCC1CCC(C(=O)ON2C(=O)CCC2=O)CC1]

To a solution of 3 (0.1 g, 0.30 mmol, 1.0 eq.) in CH₂Cl₂ (5 mL) was added TFA (0.03 mL, 0.33 mmol, 1.0 eq.). The resultant reaction mixture was stirred at room temperature for 2 h. When complete by TLC (25% EtOAc in Heptane, followed by staining with p-anisaldehyde), the reaction mixture was concentrated in vacuo to afford SAOCC as an oil (99% yield). The product was analyzed by NMR and IR.

Trifluoroacetic acid (TFA) was selected to cleave the Boc effectively. Although other acids, such as hydrochloric acid in methanol may be used, TFA cleaves Boc very effectively.

### Example 2. Condensation of SAOCC With Aldehyde/Ketone Carbonyl Groups

SAOCC was successfully condensed with benzaldehyde (an aldehyde) and acetone (a ketone) as an initial proof of concept. Additional example condensations were also prepared as follows.

### Example 2a. Condensation With p-Acetylphenylalanine

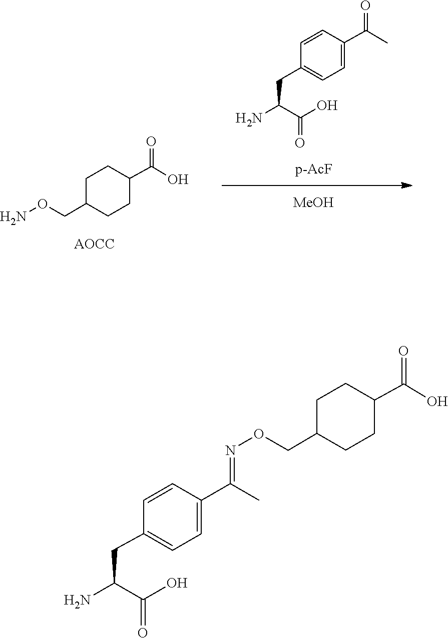 [SMILES: "MeOH" ; NOCC1CCC(C(=O)O)CC1 ; CC(=O)c1ccc(C[C@H](N)C(=O)O)cc1 ; C/C(=N\OCC1CCC(C(=O)O)CC1)c1ccc(C[C@H](N)C(=O)O)cc1]

AOCC and p-AcF (an amino acid with a ketone functionality) were stirred in methanol at 25° C. for 5 days. AOCC was selected because p-acetylphenylalanine has an amino (−NH₂) group that can react with a reactive ester such as the succinimide ester found in SAOCC. The condensation product was confirmed by TLC and NMR.

### Example 2b. Condensation With D-Glucose

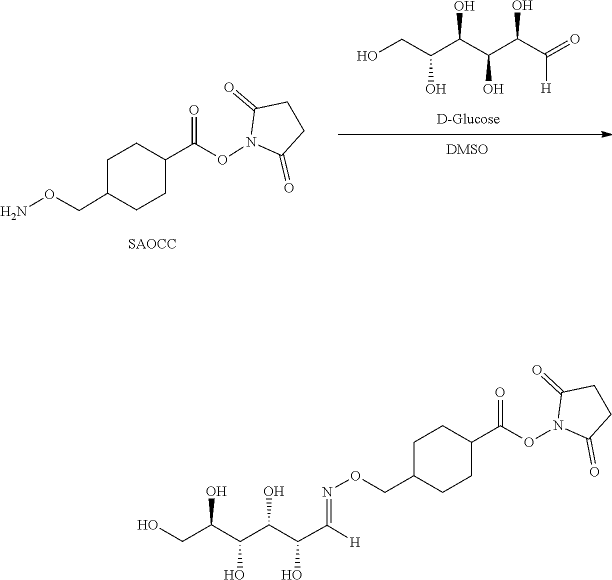 [SMILES: NOCC1CCC(C(=O)ON2C(=O)CCC2=O)CC1 ; O=C[C@H](O)[C@@H](O)[C@H](O)[C@H](O)CO ; O=C(ON1C(=O)CCC1=O)C1CCC(CO/N=C/[C@H](O)[C@@H](O)[C@H](O)[C@H](O)CO)CC1]

SAOCC and D-glucose were stirred in dimethylsulfoxide at 25° C. for 48 hours. The condensation product was confirmed by TLC and NMR. Beneficially, although D-glucose is a reducing sugar, SAOCC is able to condense with it.

### Example 2c Condensation With Sucrose

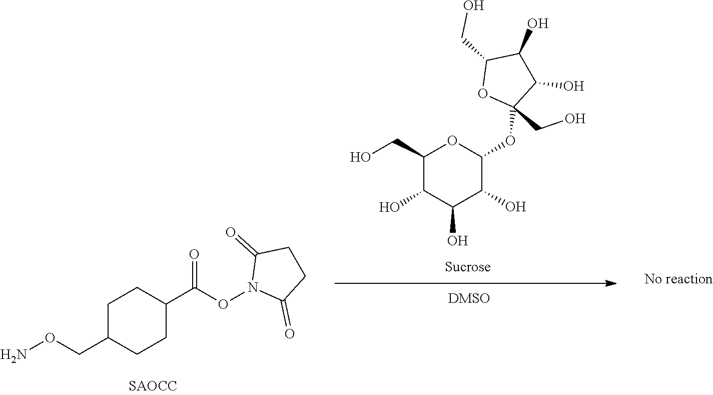

SAOCC and sucrose were stirred in dimethylsulfoxide at 25° C. for 3 days. No reaction was detected by TLC and NMR. Sucrose is a nonreducing sugar and SAOCC does not appear to condense with it. This is evidence that the SAOCC biolinker described herein is able to distinguish between reducing and nonreducing sugars.

### Example 3. Poly-ONH₂ and Poly-OPht Preparation

In some embodiments, it is beneficial to include a spacer that provides a plurality of sites to bond with more than one type of payload. Spacers such as Poly-ONH₂ provide several such alkoxyamine (aka ‘aminooxy’) sites. The Poly-ONH₂ compounds are useful in preparing antibody drug conjugates (ADC) having a high concentration and/or more than one type of therapeutic compound bound to it.

To prepare a Poly-OPht, 2-[(3-methyloxetan-3-yl)methoxy]isoindoline-1,3-dione) (shown below as compound 1) was prepared by a process outlined in Bellinghiere, A. T. et al., A facile synthesis of O-[(3-methyloxetan-3-yl)methyl]hydroxylamine, ORG. PREP. PROCED. INT., 47:232-235 (2015), which is herein incorporated by reference in its entirety. To a solution of compound 1 (1.0 g, 4.04 mmol, 1 eq.) in CH₂Cl₂ (32 mL) at 0° C. were added H₂O (0.007 g, 0.40 mmol, 0.1 eq.) followed by BF₃.OEt₂ (0.01 mL, 0.08 mmol, 0.02 eq.).

The resultant reaction mixture was allowed to reach room temperature overnight (15 h). When the TLC (50% EtOAc in Heptane) indicated reaction completion (typically 1-7 days), the reaction mixture was cooled to 0° C. and quenched with saturated NaHCO₃ (aq). Upon separating the layers, the aqueous layer was extracted with CH₂Cl₂ (3×15 mL). The combined organic layer was then washed with saturated NaHCO₃ (aq) (2×30 mL), H₂O (2×30 mL) and saturated NaCl (aq) (2×30 mL). The organic layer was dried over Na₂SO₄ and the solvent was removed in vacuo to afford Poly-OPht (shown below as compound 2) as a solid (87% yield). The degree of polymerization (n) was determined by NMR.

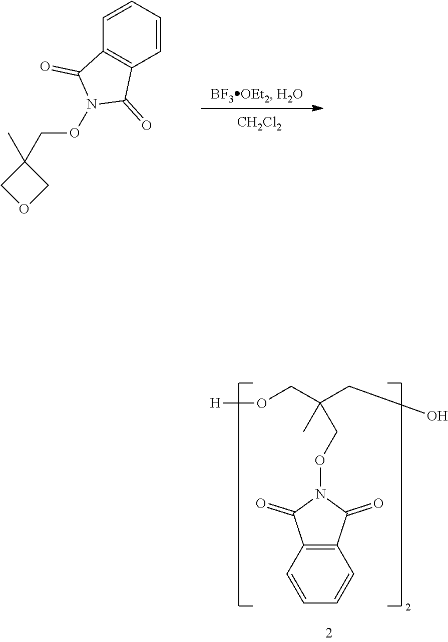

Next, to a solution of compound 2 (1.34 g, 0.89 mmol, 1 eq.) in CH₂Cl₂ (25 mL) and EtOH (50 mL) at 25° C. was added H₂NNH₂.H₂O (0.67 mL, 13.3 mmol, 15 eq.). The resultant reaction mixture was allowed to stir at 25° C. for 24 h. After removal of solvents in vacuo, the residue was diluted with CH₂Cl₂ (100 mL). The resultant slurry was stirred with 1M NaOH (30 mL) until two homogeneous layers were obtained (<5 min at 25° C.). The layers were separated and the organic layer was washed with 1M NaOH (30 mL), H₂O (2×50 mL) and saturated NaCl (aq) (50 mL) and dried over Na₂SO₄. Removal of solvents in vacuo afforded Poly-ONH₂ as an oil (75% yield), as shown below. The product was analyzed by NMR.

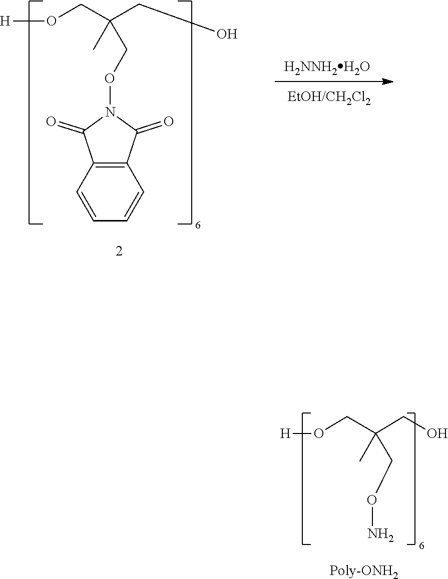

The embodiments being thus described, it will be obvious that the same may be varied in many ways. Such variations are not to be regarded as a departure from the spirit and scope of the disclosure and all such modifications are intended to be included within the scope of the following claims.

What is claimed is:

1. A method of synthesizing a heterofunctional biolinker comprising:
(a) converting a cyclohexane carboxylic acid according to the structure 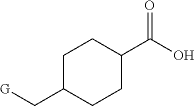
into a carboxylic acid alkyl halide, wherein G is any group that be converted into a leaving group (e.g., a halide);
(b) reacting the carboxylic acid alkyl halide with a protected hydroxylamine to yield a a protected alkoxyamine;
(c) converting a carboxyl group on the protected alkoxyamine into a reactive ester to yield a protected alkoxyamine reactive ester;
(d) deprotecting the protected alkoxyamine reactive ester;
(e) optionally, adding a spacer; and
(f) yielding a heterofunctional biolinker according to the structure: 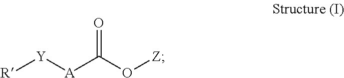
wherein R′ is —ONH₂; wherein A is a spacer; wherein Y is absent or an additional spacer; and wherein Z is a carboxylic acid activator that provides a reactive ester site.

2. The method of claim 1, wherein the spacer is a compound according to the formula:
G-A-Nu
wherein G is a group that can be converted into an aminooxy comprising —OH, -Suc, —OPht, or an S_{N}2 leaving group; A is one or more monomers; and Nu is a nucleophilic group comprising —OH, —SH, or —NH₂.

3. The method of claim 1, wherein the spacer is a compound according to the formula:
G-A-LG
wherein G is a group that can be converted into an aminooxy comprising —OH, -Suc, —OPht, or an S_{N}2 leaving group; A is one or more monomers; and LG is a leaving group comprising a tosylate, mesylate, a triflate, a triphenyl phosphate, fluoride, chloride, bromide, iodide, water, alcohol, nitrate, phosphate, thioester, amine, ammonium, carboxylate, phenoxide group, or a combination thereof.

4. The method of claim 3, wherein the S_{N}2 leaving group is a dinitrogen, dialkyl ether, perfluyoroalkylsulfonate, tosylate, mesylate, a triflate, a triphenyl phosphate, fluoride, chloride, bromide, iodide, water, alcohol, nitrate, phosphate, thioester, amine, ammonium, carboxylate, phenoxide, or a combination thereof.

5. The method of claim 1, wherein the spacer and/or the additional spacer comprises:
(i) a substituted or unsubstituted, linear or branched C₁-C₂₀ hydrocarbon;
(ii) a polyethylene glycol according to the structure 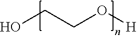
wherein n is an integer between 1 and 10,000;
(iii) an acyclic diol according to the structure 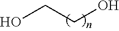
wherein n is an integer between 1 and 10,000;
(iv) a cyclic diol according to the structure 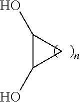
wherein n is an integer between 1 and 10,000;
(v) a poly-ONH₂ according to the structure 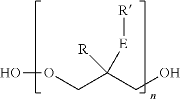
wherein n is an integer between 1 and 10,000; R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

6. The method of claim 1, wherein the spacer and/or the additional spacer comprises:
(i) a substituted or unsubstituted, linear or branched C₁-C₂₀ hydrocarbon;
(ii) a halogen polyethylene glycol according to the structure 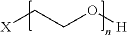
wherein X is a fluoride, chloride, bromide or iodide; n is an integer between 1 and 10,000;
(iii) a halogenated alcohol according to the structure 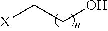
wherein X is a fluoride, chloride, bromide or iodide; n is an integer between 1 and 10,000;
(iv) a halogenated cyclic compound according to the structure 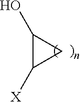
wherein X is a fluoride, chloride, bromide or iodide; n is an integer between 1 and 10,000;
(v) a poly-ONH₂ according to the structure 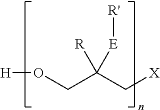
wherein n is an integer between 1 and 10,000; R′ is —ONH₂; X is a fluoride, chloride, bromide or iodide; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

7. The method of claim 1, wherein the carboxylic acid alkyl halide is a compound according to the formula: 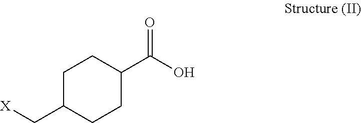
wherein X is a halide.

8. The method of claim 7, wherein the carboxylic acid alkyl halide comprises an alkyl fluoride, alkyl chloride, alkyl bromide, or alkyl iodide.

9. The method of any one of claims 8, wherein the protected hydroxylamine is a compound according to the structure: 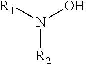
wherein R₁ is H and wherein R₂ is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

10. The method of claim 9, wherein the protected hydroxylamine is phenyl hydroxylamine, tolyl hydroxylamine, 2-iodophenyl hydroxylamine, N-benzyl hydroxylamine, N-methoxycarbonyl hydroxylamine, N-ethoxycarbonyl hydroxylamine, N-Boc hydroxylamine, N-CBz hydroxylamine, N-FMoc hydroxylamine, N-hydroxysuccinimide, N-hydroxyphthalimide or a combination thereof.

11. The method of claim 10, wherein the protected alkoxyamine is a compound according to the structure: 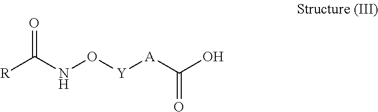
wherein R is independently selected from H, —OC(CH₃)₃, —OCH₂R where R is H, methyl, phenyl or fluorenyl, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof, the spacer A is independently selected from substituted or unsubstituted, linear or branched C₁-C₂₀ alkyl, cycloalkyl, aryl, cyclohexyl, or phenyl group or a combination thereof; and the additional spacer Y is independently selected from a substituted or unsubstituted, linear or branched C₁-C₂₀ hydrocarbon, polyethylene glycol (PEG), or a combination thereof.

12. The method of claim 11, wherein the protected alkoxyamine reactive ester is a compound according to the structure: 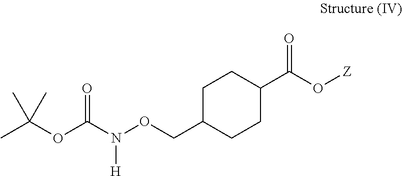
wherein Z is a carboxylic acid activator that provides a reactive ester site.

13. The method of claim 12, wherein the heterofunctional biolinker comprises a label L according to the structures: 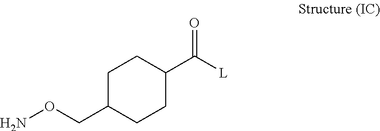
wherein L is the label; or 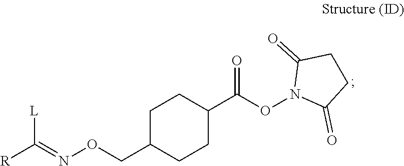
wherein L is the label.

14. The method of claim 13, wherein the label comprises horseradish peroxidase, a fluorescent dye, a metal, an epitope tag, a protein, an antibody, or a combination thereof.

15. The method of claim 13, wherein the heterofunctional biolinker is a compound according to the structure:

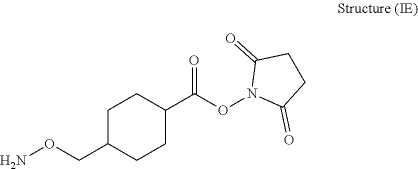 [SMILES: NOCC1CCC(C(=O)ON2C(=O)CCC2=O)CC1]

16. A method of making an antibody drug conjugate compound comprising an antibody covalently attached by a heterofunctional biolinker to a drug moiety, the compound having the formula:
Ab-(H-(D)x)y
or a pharmaceutically acceptable salt or solvate thereof; wherein Ab is an antibody or an antigen binding fragment thereof; wherein D is the drug moiety, wherein x is an integer from 1 to 20, y is an integer from 1 to 10,000, and wherein H is a heterofunctional biolinker according to the structure: 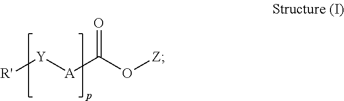
wherein R′ is —ONH₂ or absent; wherein A is a spacer; wherein Y is absent or an additional spacer; wherein Z is a carboxylic acid activator that provides a reactive ester site; and wherein p is an integer from 1 to 10,000;
wherein the method comprises:
reacting Ab with the heterofunctional biolinker to form an antibody-biolinker intermediate and reacting the antibody-biolinker intermediate with the drug moiety D to form the antibody drug conjugate; or
reacting the drug moiety D with the heterofunctional biolinker to form a drug-biolinker intermediate, and then reacting the drug-biolinker intermediate with the antibody to form the antibody drug conjugate.

17. The method of claim 16, wherein the antibody drug conjugate is a compound according to the structure: 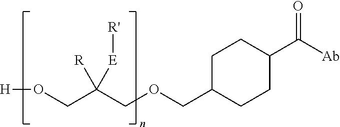
wherein R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; n is an integer between 1 and 10,000; and Ab is an antibody or an antigen binding fragment thereof.

18. The method of claim 16, wherein the antibody drug conjugate is a compound according to the formula: 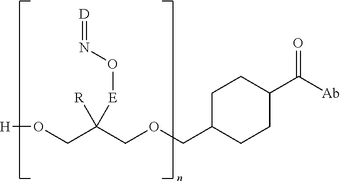
wherein Ab is an antibody or an antigen binding fragment thereof; n is an integer between 1 and 1,000; D is the drug moiety; R is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; and E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

19. The method of claim 16, wherein the drug moiety comprises one drug or therapeutic agent; or wherein the drug moiety comprises two or more different drugs or therapeutic agents.

20. The method of claim 16, wherein the drug moiety comprises a V-ATPase inhibitor, HSP90 inhibitor, IAP inhibitor, mTor inhibitor, microtubule stabilizer, microtubule destabilizer, auristatin, dolastatin, maytansinoid, methionine aminopeptidase (MetAP), CRM1 inhibitor, DPPIV inhibitor, phosphoryl transfer reaction transfer inhibitor, protein synthesis inhibitor, kinase inhibitor, CDK2 inhibitor, CDK9 inhibitor, proteasome inhibitor, kinesin inhibitor, HDAC inhibitor, DNA damaging agent, DNA alkylating agent, DNA intercalator, DNA minor groove binder, checkpoint inhibitor, immune checkpoint inhibitor, cancer growth blocker, angiogenesis inhibitor, DHFR inhibitor, or a combination thereof.

21. The method of claim 16 wherein the drug moiety comprises a biophysical probe, a fluorophore, a spin label, an infrared probe, an affinity probe, a chelator, a spectroscopic probe, a radioactive probe, a lipid molecule, a polyethylene glycol, a polymer, a spin label, DNA, RNA, a protein, a peptide, a surface, an antibody, an antibody fragment, a nanoparticle, a quantum dot, a liposome, a PLGA particle, a saccharide or a polysaccharide.

22. A heterofunctional biolinker made by the process of claim 1.

23. An antibody drug conjugate made by the process of claim 16.

24. A heterofunctional biolinker composition comprising:
a heterofunctional biolinker according to the structure: 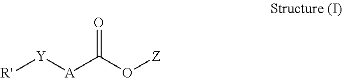
wherein R′ is —ONH₂ or absent; wherein A is a spacer; wherein Y is absent or an additional spacer; and wherein Z is a carboxylic acid activator that provides a reactive ester site.

25. The composition of claim 24, wherein the spacer and/or the additional spacer is a compound according to the formula:
G-A-Nu
wherein G is a group that can be converted into an aminooxy comprising —OH, -Suc, —OPht, or an S_{N}2 leaving group; A is one or more monomers; and Nu is a nucleophilic group comprising —OH, —SH, or —NH₂.

26. The composition of claim 24, wherein the spacer and/or the additional spacer is a compound according to the formula:
G-A-LG
wherein G is a group that can be converted into an aminooxy comprising —OH, -Suc, —OPht, or a combination thereof; A is one or more monomers; and LG is a leaving group.

27. The composition of claim 25, wherein the S_{N}2 leaving group is a dinitrogen, dialkyl ether, perfluoroalkylsulfonate, tosylate, mesylate, a triflate, a triphenyl phosphate, fluoride, chloride, bromide, iodide, water, alcohol, nitrate, phosphate, thioester, amine, ammonium, carboxylate, phenoxide, or a combination thereof.

28. The composition of claim 24, wherein the spacer and/or the additional spacer comprises:
(i) a substituted or unsubstituted, linear or branched C₁-C₂₀ hydrocarbon;
(ii) a polyethylene glycol according to the structure 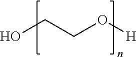
wherein n is an integer between 1 and 10,000;
(iii) an acyclic diol according to the structure 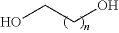
wherein n is an integer between 1 and 10,000;
(iv) a cyclic diol according to the structure 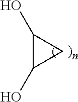
wherein n is an integer between 1 and 10,000;
(v) a poly-ONH2 according to the structure 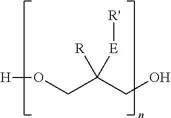
wherein n is an integer between 1 and 10,000; R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

29. The composition of claim 24, wherein the spacer and/or the additional spacer comprises:
(i) a substituted or unsubstituted, linear or branched C₁-C₂₀ hydrocarbon;
(ii) a halogen polyethylene glycol according to the structure 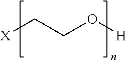
wherein n is an integer between 1 and 10,000 and X is a fluoride, chloride, bromide or iodide;
(iii) a halogenated alcohol according to the structure 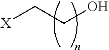
wherein n is an integer between 1 and 10,000 and X is a fluoride, chloride, bromide or iodide;
(iv) a halogenated cyclic compound according to the structure 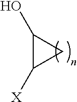
wherein n is an integer between 1 and 10,000 and X is a fluoride, chloride, bromide or iodide;
(v) a poly —ONH₂ according to the structure 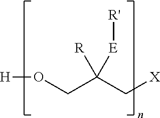
wherein n is an integer between 1 and 10,000; X is a fluoride, chloride, bromide or iodide; R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof;
(vi) or a combination thereof.

30. The composition of claim 24, wherein the heterofunctional biolinker further comprises an antibody or antigen binding fragment thereof Ab and is a compound according to the structure: 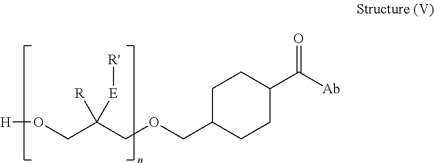
wherein R′ is —ONH₂; R is H or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; n is an integer between 1 and 10,000; and Ab is the antibody or an antigen binding fragment thereof.

31. The composition of claim 24, wherein the heterofunctional biolinker further comprises an antibody or an antigen binding fragment Ab and a drug moiety D, and is a compound according to the structure: 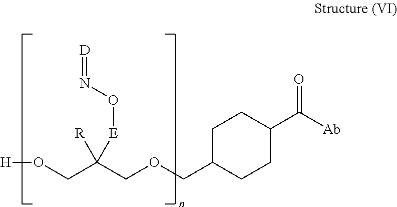
wherein Ab is the antibody or an antigen binding fragment thereof; n is an integer between 1 and 1,000; D is the drug moiety; R is H, or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof; and E is absent or a substituted or unsubstituted linear or branched C₁-C₆ alkyl, C₃-C₇, cycloalkyl, cycloalkyl, aryl, arylalkyl, heterocyclyl, heterocyclylalkyl, or a combination thereof.

32. The composition of claim 30, wherein the drug moiety comprises one drug or therapeutic agent; or wherein the drug moiety comprises two or more different drugs or therapeutic agents.

33. The composition of claim 24, wherein the composition comprises a mixture of heterofunctional biolinkers.

34. The composition of claim 24, further comprising a pharmaceutically acceptable diluent, carrier, excipient, or a combination thereof.

35. A method of treating a disease in a patient in need thereof comprising:
administering to the patient a therapeutically effective amount of the composition of claim 24.

36. The method of claim 35, wherein the composition is administered in a concentration of about 0.1 to about 100 mg/kg of the patient weight per dose.

37. The method of claim 35, wherein the composition is administered at intervals of between one week to four weeks.

38. The method of claim 35, wherein the disease is cancer, HIV, an autoimmune disease, a cardiovascular disease, or a combination thereof.

39. The method of claim 38, wherein the cancer is leukemia, lymphoma, breast cancer, cervical cancer, bladder cancer, or multiple myeloma.
